SEQUENCE LISTING (24 sequences)

SEQ ID NO: 1-4 - skipped (4 entries)

SEQ ID NO: 5 - DNA, 19 bp, synthetic construct
  source 1..19: mol_type = other DNA
tcgtatctaatgccatgta

SEQ ID NO: 6 - DNA, 19 bp, synthetic construct
  source 1..19: mol_type = other DNA
tcgtatctaacgccacgta

SEQ ID NO: 7 - DNA, 48 bp, synthetic construct
  source 1..48: mol_type = other DNA
accatctcatccctgcgtgtctccgactgcacacatcctgcatgtgat

SEQ ID NO: 8 - DNA, 48 bp, synthetic construct; identical to SEQ ID NO: 7
  source 1..48: mol_type = other DNA

SEQ ID NO: 9 - DNA, 98 bp, synthetic construct
  source 1..98: mol_type = other DNA
tcacatgcaggatgtgtgcannnatagtccaccgtcactgtcggtcgtagagctcagaacggattggcgctagactctgacgtgttgatcctcgaagc

SEQ ID NO: 10 - DNA, 98 bp, synthetic construct
  source 1..98: mol_type = other DNA
gtagtctaacgctcggtgnnncagatgtacgacaatgatcacttagtcacttattgggtcacggtgtggcttcgaggatcaacacgtcagagtctagc

SEQ ID NO: 11 - DNA, 46 bp, synthetic construct
  source 1..46: mol_type = other DNA
  misc_feature 45: note = phosphorylated nucleotide base
ctcagctctctatgggcagtcggtgatcaccgagcgttagactact

SEQ ID NO: 12 - DNA, 95 bp, synthetic construct
  source 1..95: mol_type = other DNA
tcacatgcaggatgtgtgcaggaaaatcccccccatggcagtgcaaacatgcaaaaggcattggcgctagactttgggcttttgaccttgcaagc

SEQ ID NO: 13 - DNA, 94 bp, synthetic construct
  source 1..94: mol_type = other DNA
gatgggaattggccctggcaatgtgcaagctttccttttttgttcatttttccccattcgggtggcttgcaaggtcaaaagcccaaagtctagc

SEQ ID NO: 14 - DNA, 46 bp, synthetic construct
  source 1..46: mol_type = other DNA
  misc_feature 45: note = phosphorylated nucleotide base
ctcagctctctatgggcagtcggtgatcagggccaattcccatcat

SEQ ID NO: 15 - DNA, 48 bp, synthetic construct
  source 1..48: mol_type = other DNA
tccatctcatccctgcgtgtctccgactgcacacatcctgcatgtgat

SEQ ID NO: 16 - DNA, 98 bp, synthetic construct
  source 1..98: mol_type = other DNA
tcacatgcaggatgtgtgcatccaaatcccccccatggcagtgcaaagatgcaaaaggcattggcgctgctagactttgggattttgaccttgaaagc

SEQ ID NO: 17 - DNA, 95 bp, synthetic construct
  source 1..95: mol_type = other DNA
  misc_feature 94: note = phosphorylated nucleotide base
tgatgggaattggccctggcaatgtggaagctttccttttttgttcatttttccccattcgggtggctttcaaggtcaaaatcccaaagtctagc

SEQ ID NO: 18 - DNA, 46 bp, synthetic construct; identical to SEQ ID NO: 14
  source 1..46: mol_type = other DNA
  misc_feature 45: note = phosphorylated nucleotide base

SEQ ID NO: 19 - DNA, 166 bp, synthetic construct
  source 1..166: mol_type = other DNA
gtagtctaacgctcggtgnnncagatgtacgacaatgatcacttagtcacttattgggtcacggtgtggcttcgaggatcaacacgtcagagtctagcgccaatccgttctgagctctacgaccgacagtgacggtggactatnnntgcacacatcctgcatgtga

SEQ ID NO: 20 - DNA, 45 bp, synthetic construct
  source 1..45: mol_type = other DNA
gtagtctaacgctcggtgatcaccgactgcccatagagagctgag

SEQ ID NO: 21 - DNA, 83 bp, synthetic construct
  source 1..83: mol_type = other DNA
tagcggtcatggtannnnnnncnnnnnncgnnnnncgnnnnnnnntgnnnnnncgnnnnnnnnncnnnnnnntgnnnnnnnnn

SEQ ID NO: 22 - DNA, 83 bp, synthetic construct
  source 1..83: mol_type = other DNA
tagcggtcatggtannnnnnncnnnnnntgnnnnntgnnnnnnnntgnnnnnntgnnnnnnnnncnnnnnnntgnnnnnnnnn

SEQ ID NO: 23 - DNA, 83 bp, synthetic construct
  source 1..83: mol_type = other DNA
tagcggtcatggtannnnnnncnnnnnncgnnnnncgnnnnnnnncgnnnnnncgnnnnnnnnncnnnnnnncgnnnnnnnnn

SEQ ID NO: 24 - DNA, 83 bp, synthetic construct
  source 1..83: mol_type = other DNA
tagcggtcatggcannnnnnncnnnnnncgnnnnncgnnnnnnnncgnnnnnncgnnnnnnnnncnnnnnnncgnnnnnnnnn

(19) United States
(12) Patent Application Publication
(10) Pub. No.: US 2025/0027154 A1
(43) Pub. Date: Jan. 23, 2025

(54) METHODS AND COMPOSITIONS FOR SIMULTANEOUSLY SEQUENCING A NUCLEIC ACID TEMPLATE SEQUENCE AND COPY SEQUENCE

(71) Applicant: ULTIMA GENOMICS, INC., Fremont, CA (US)

(72) Inventors: Zohar SHIPONY, Rehovot (IL); Florian OBERSTRASS, Redwood City, CA (US); Doron LIPSON, Ramat Hasharon (IL); Eti MEIRI, Shoham (IL); Tommie Lloyd LINCECUM, JR., Carlsbad, CA (US); Daniel MAZUR, San Diego, CA (US); Omer BARAD, Mazkeret Batya (IL)

(21) Appl. No.: 18/803,028

(22) Filed: Aug. 13, 2024

Related U.S. Application Data
(63) Continuation of application No. PCT/US2023/063064, filed on Feb. 22, 2023.
(60) Provisional application No. 63/313,217, filed on Feb. 23, 2022.

(51) Int. Cl. C12Q 1/6876; C12Q 1/6869

(52) U.S. Cl. CPC C12Q 1/6876; C12Q 1/6869; C12Q 2600/154

(57) ABSTRACT

Nucleic acid molecule constructs, methods of making such construct, and methods of sequencing using such constructs are described herein. The nucleic acid molecule constructs can include a template sequence and a copy of the template sequence, along with a sequencing primer region associated with the template sequence and a copy of the sequencing primer region associated with the copy of the template sequence, which allows the template sequence and the copy of the template sequence to be simultaneously sequenced. Also described are methods of using the nucleic acid molecule constructs for methylation sequencing.

### CROSS-REFERENCE TO RELATED APPLICATIONS

This application is a continuation of International Application No. PCT/US2023/063064, filed Feb. 22, 2023, which claims the priority benefit to U.S. Provisional Application No. 63/313,217, filed on Feb. 23, 2022: the entire contents of which are incorporated herein by reference for all purposes.

### REFERENCE TO AN ELECTRONIC SEQUENCE LISTING

The instant application contains a Sequence Listing which has been submitted electronically in XML format and is hereby incorporated by reference in its entirety. Said XML copy, created on Feb. 22, 2023, is named Ultima 51024-777.301 SL.XML and is 22,094 bytes in size.

### FIELD OF THE INVENTION

Described herein are methods of sequencing a polynucleotide, including methods for determining a methylation profile for the polynucleotide, as well as nucleic acid molecule constructs used for sequencing.

### BACKGROUND

Next-generation sequencing (NGS) methods allow for high throughput sequencing of polynucleotides, giving insight into genetic profiles of patients and cancers. Methylation patterns on certain genes can be associated with certain aspects of a cancer, for example responsiveness to certain therapies or cancer driving mechanisms. However, NGS sequencing alone does not provide a methylation profile.

Chemical and enzymatic processes can selectively modify methylated or non-methylated cytosine bases. For example, treating a 5-methylated cytosine (5mC) with bisulfate can convert the methylated cytosine to a uracil base. This selective conversion can be used to identify methylated cytosine nucleotides in a target sequence. However, such a modification disrupts the nucleotide sequence, making it challenging to map a location of a methylated cytosine to a particular locus within the subject genome.

### BRIEF SUMMARY OF THE INVENTION

Described herein is a composition, comprising: an oligonucleotide set, comprising a first oligonucleotide, a second oligonucleotide, a third oligonucleotide, and a fourth oligonucleotide, wherein: a 3′ portion of the first oligonucleotide is hybridized to a 5′ portion of the second oligonucleotide, wherein 3′ portion of the first oligonucleotide comprises a sequencing primer region, a 3′ portion of the second oligonucleotide is hybridized to a 3′ portion of the third oligonucleotide, and a 5′ portion of the third oligonucleotide is hybridized to a 3′ portion of the fourth oligonucleotide.

In some embodiments, a 3′ terminus of the first oligonucleotide is coupled to a 5′ terminus of a first strand of a template nucleic acid molecule, a 5′ terminus of the second oligonucleotide is coupled to a 3′ terminus of a second strand of the template nucleic acid molecule, wherein the second strand is hybridized to the first strand, a 5′ terminus of the third oligonucleotide is coupled to a 3′ terminus of the first strand, and a 3′ terminus of the fourth oligonucleotide is coupled to a 5′ terminus of the second strand. In some embodiments, the first strand of the template nucleic acid molecule comprises a first template sequence; and the second strand of the template nucleic acid molecule comprises a second template sequence, wherein the second template sequence is a reverse complement of the first template sequence. In some embodiments, 3′ portion of the first oligonucleotide comprises a barcode sequence positioned between the sequencing primer region and the 3′ terminus of the first oligonucleotide. In some embodiments, a 5′ portion of the first oligonucleotide comprises a forward amplification primer region, and a 5′ portion of the fourth oligonucleotide comprises a reverse amplification primer region.

Further described herein is a composition, comprising: a nucleic acid molecule comprising a first construct strand and a second construct strand, wherein: the first construct strand comprises, from 5′ to 3′, a first sequencing primer region, a first template sequence, a first nucleic acid linker, a copy of the first sequencing primer region, and a single copy of the first template sequence; and the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a single copy of the second template sequence, and a copy of the second sequencing primer region.

Also described herein is a composition, comprising: a nucleic acid molecule comprising a first construct strand and a second construct strand, wherein: the first construct strand comprises, from 5′ to 3′, a first sequencing primer region, a first template sequence, a first nucleic acid linker, a copy of the first sequencing primer region, wherein substantially all cytosine bases in the copy of the first sequencing primer region are methylated, and a copy of the first template sequence, wherein substantially all cytosine bases in the copy of the first template sequence are methylated; and the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, wherein substantially all cytosine bases in the copy of the second template sequence are methylated, and a copy of the second sequencing primer region, wherein substantially all cytosine bases in the copy of the second sequencing primer region are methylated.

Also described is a method, comprising: (a) providing: a template nucleic acid molecule comprising a first strand comprising a first template sequence and a second strand comprising a second template sequence, wherein the first template sequence is a reverse complement of the second template sequence and wherein the first strand is hybridized to the second strand; and an oligonucleotide set, comprising a first oligonucleotide, a second oligonucleotide, a third oligonucleotide, and a fourth oligonucleotide, wherein: a 3′ portion of the first oligonucleotide hybridizes to a 5′ portion of the second oligonucleotide, wherein the 3′ portion of the first oligonucleotide comprises a sequencing primer region, a 3′ portion of the second oligonucleotide hybridizes to a 3′ portion of the third oligonucleotide, and a 5′ portion of the third oligonucleotide hybridizes to a 3′ portion of the fourth oligonucleotide; and (b) ligating: a 3′ terminus of the first oligonucleotide to a 5′ terminus of the first strand, a 5′ terminus of the second oligonucleotide to a 3′ terminus of the second strand, a 5′ terminus of the third oligonucleotide to a 3′ terminus of the first strand, and a 3′ terminus of the fourth oligonucleotide to a 5′ terminus of the second strand.

The method may further include crosslinking the second oligonucleotide to the third oligonucleotide. In some embodiments, the crosslinking is a reversible crosslinking. In some embodiments, the second oligonucleotide is crosslinked to the third oligonucleotide before the ligating. In some embodiments, the second oligonucleotide is crosslinked to the third oligonucleotide after the ligating.

In some embodiments of the described method the method further comprises: (c) performing extension reactions, comprising: extending the 3′ terminus of the second oligonucleotide using, in order, a portion of the third oligonucleotide, the first strand, and the first oligonucleotide as a template, extending the 3′ terminus of the third oligonucleotide using, in order, a portion of the second oligonucleotide, the second strand, and the fourth oligonucleotide as a template. In some embodiments, the extension reactions generate a nucleic acid molecule comprising a first construct strand and a second construct strand, wherein: the first construct strand comprises, from 5′ to 3′, a first sequencing primer region, a first template sequence, a first nucleic acid linker, a copy of the first sequencing primer region, and a copy of the first template sequence; and the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, and a copy of the second sequencing primer region. In some embodiments, the extension reactions are performed in the presence of a nucleotide reagent comprising methylated cytosine bases and substantially no unmethylated cytosine bases, and wherein substantially all cytosine bases in the copy of the first sequencing primer region, the copy of the first template sequence, the copy of the sequence sequencing primer region, and the copy of the second template sequence are methylated. In some embodiments, the sequencing primer region is free of cytosine bases.

In some embodiments, the first nucleic acid linker or the second nucleic acid linker is about 30 bases in length to about the length of the first template sequence or the second template sequence. In some embodiments, the first nucleic acid linker or the second nucleic acid linker is between about 20% and about 100% of a length of the first template sequence or the second template sequence. In some embodiments, the first nucleic acid linker and the second nucleic acid linker each have a known sequence.

In some embodiments, 3′ portion of the first oligonucleotide further comprises a barcode sequence. In some embodiments, the barcode sequence is positioned between the sequencing primer region and the 3′ terminus of the first strand. In some embodiments, the barcode sequence comprises a unique molecular identifier. In some embodiments, the barcode sequence comprises a sample barcode.

In some embodiments, 3′ portion of the first oligonucleotide further comprises a preamble sequence. In some embodiments, the preamble sequence is positioned between the sequencing primer region and the 3′ terminus of the first strand.

Further described is a nucleic acid molecule construct made according to the above.

The described method may further include generating differential profile data comprising: first sequencing data, comprising a nucleic acid sequence corresponding to the first template sequence or the second template sequence; and second sequencing data, comprising a nucleic acid sequence corresponding to the copy of the first template sequence or the copy of the second template sequence. In some embodiments, the first sequencing data and the second sequencing data of the differential profile data are obtained from a same first strand sequencing read or a same second strand sequencing read. In some embodiments, the method further comprises filtering library sequencing data to remove sequencing reads associated with a difference between the first sequencing data and the second sequencing data. In some embodiments, the differential profile data comprises methylation data, and the method further comprises identifying a location of one or more methylated or unmethylated cytosine residues in the template nucleic acid molecule.

In some embodiments, the method further comprises generating sequencing data, comprising: hybridizing sequencing primers to the first sequencing primer region and to the copy of the first sequencing primer region on the first construct strand to form a hybridized template; and generating sequencing data from the first template sequence and the first copy portion simultaneously, comprising extending the sequencing primers by, for each of a plurality of sequencing flow steps, (i) providing, to the hybridized template, labeled nucleotides of a single base type, and (ii) detecting a signal indicating incorporation of a labeled nucleotide into the extending sequencing primers.

In some embodiments, the method further comprises generating sequencing data, comprising: hybridizing sequencing primers to the second sequencing primer region and to the copy of the second sequencing primer region on the second construct strand to form a hybridized template; and generating sequencing data from the second template sequence and the second copy portion simultaneously, comprising extending the sequencing primers by, for each of a plurality of sequencing flow steps, (i) providing, to the hybridized template, labeled nucleotides of a single base type, and (ii) detecting a signal indicating incorporation of a labeled nucleotide into the extending sequencing primers.

Also described herein is a method, comprising: (a) providing: a template nucleic acid molecule comprising a first strand comprising a first template sequence and a second strand comprising a second template sequence, wherein the first template sequence is a reverse complement of the second template sequence; and an oligonucleotide set, comprising a first oligonucleotide, a second oligonucleotide, a third oligonucleotide, and a fourth oligonucleotide, wherein: a 3′ portion of the first oligonucleotide hybridizes to a 5′ portion of the second oligonucleotide, wherein 3′ portion of the first oligonucleotide comprises a first sequencing primer region, a 3′ portion of the second oligonucleotide hybridizes to a 3′ portion of the third oligonucleotide, and a 5′ portion of the third oligonucleotide hybridizes to a 3′ portion of the fourth oligonucleotide: (b) ligating: a 3′ terminus of the first oligonucleotide to a 5′ terminus of the first strand, a 5′ terminus of the second oligonucleotide to a 3′ terminus of the second strand, a 5′ terminus of the third oligonucleotide to a 3′ terminus of the first strand, and a 3′ terminus of the fourth oligonucleotide to a 5′ terminus of the second strand; (c) performing extension reactions in the presence of a nucleotide reagent comprising methylated cytosine bases, comprising: extending the 3′ terminus of the second oligonucleotide using, in order, a portion of the third oligonucleotide, the first strand, and the first oligonucleotide as a template, extending the 3′ terminus of the third oligonucleotide using, in order, a portion of the second oligonucleotide, the second strand, and the fourth oligonucleotide as a template, thereby generating a nucleic acid molecule comprising a first construct strand and a second construct strand, wherein: the first construct strand comprises, from 5′ to 3′, a first sequencing primer region, a first template sequence, a first nucleic acid linker, a copy of the first sequencing primer region, and a copy of the first template sequence; and the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, and a copy of the second sequencing primer region; (d) sequencing the first strand, comprising: hybridizing sequencing primers to the first sequencing primer region and to the copy of the first sequencing primer region on the first strand, or to the second sequencing primer region and to the copy of the second sequencing primer region on the second strand, to form a hybridized template; generating first sequencing data for the copy of the first template sequence or the copy of the second template sequence, comprising extending the sequencing primers by, for each of a plurality of sequencing flow steps according to a first flow order, (i) providing, to the hybridized template, labeled nucleotides of a single base type, and (ii) detecting a signal indicating incorporation of a labeled nucleotide into the extending sequencing primers; and generating second sequencing data for the first template sequence or the second template sequence, comprising extending the sequencing primers by, for each of a plurality of sequencing flow steps according to a second flow order, (i) providing, to the hybridized template, labeled nucleotides of a single base type, and (ii) detecting a signal indicating incorporation of a labeled nucleotide into the extending sequencing primers, wherein the first sequencing data and the second sequencing data are generated simultaneously.

### BRIEF DESCRIPTION OF THE DRAWINGS

FIG. 1 illustrates an exemplary embodiment of a nucleic acid construct described herein, in accordance with some embodiments.

FIG. 2 shows an exemplary oligonucleotide set, according to some embodiments.

FIG. 3 shows an exemplary method of making a nucleic acid construct, according to some embodiments.

FIG. 4 shows an exemplary method for targeted enrichment of a CpG site according to some embodiments

FIG. 5 illustrates exemplary methylation status data that may be obtained using the method described herein, in accordance with some embodiments.

FIG. 6A illustrates an exemplary method for obtaining methylation data (and/or sequencing data) for a nucleic acid molecule.

FIG. 6B illustrates an exemplary method for generating methylation data (and/or sequencing data) for a nucleic acid molecule.

### DETAILED DESCRIPTION OF THE INVENTION

### Definitions

As used in this specification and the appended claims, the singular forms “a”, “an”, and “the” include plural references unless the context clearly dictates otherwise.

As used herein, the term “about” a number refers to that number plus or minus 10% of that number. The term “about” when used in the context of a range refers to that range minus 10% of its lowest value and plus 10% of its greatest value.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “nucleotide” refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a nucleotide analog that is a modified, synthesized, or engineered nucleotide). A naturally occurring nucleotide may include a canonical base (e.g., A, C, G, T, or U). A nucleotide analog may not be naturally occurring or may include a non-canonical base (e.g., an alternative base). The nucleotide analog may include a modified polyphosphate chain (e.g., triphosphate coupled to a fluorophore). The nucleotide analog may comprise a label. The nucleotide analog may be terminated (e.g., reversibly terminated). In some cases, the nucleotide analog may not be terminated (e.g., multiple nucleotides may be incorporated in a homopolymer region). Nucleotide analogs that may be used in accordance with embodiments of this disclosure are described, for example, in United States Patent Publication No. 2021/0230669, which is hereby incorporated by reference in its entirety.

Any reference to “or” herein is intended to encompass “and/or” unless otherwise stated.

A “copy” of a nucleotide sequence refers to a replication of the canonical nucleobase sequence (A, C, G, and T (and/or U)), without regard to the methylation status of any nucleobase sequence, unless otherwise indicated.

The term “label” refers to a detectable label that emits a signal, or reduces or enhances a signal, where the signal can be detected (e.g., a luminescent signal, a fluorescent signal, a phosphorescent signal, or a radioactive signal). The signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl) phosphine (TCEP), or tris(hydroxypropyl)phosphine (THP)), or enzymatically cleavable (e.g., via an esterase, lipase, peptidase, or protease).

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3): 443-53): Smith-Waterman (see, e.g., Smith, Temple F.; and Waterman, Michael S., “Identification of Common Molecular Subsequences” (1981) Journal of Molecular Biology 147:195-197): or BLAST (Basic Local Alignment Search Tool: see, e.g., Altschul S F, Gish W, Miller W, Myers E W, Lipman D J, “Basic local alignment search tool” (1990) J Mol Biol 215 (3): 403-410).

As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical”.

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Nucleic Acid Constructs

The nucleic acid constructs (also referred to as “nucleic acid molecule constructs” or “constructs”) provided herein includes a nucleic acid construct strand that has a template sequence and a copy of the template sequence (e.g., a single copy of the template sequence, in addition to the template sequence itself). As further described herein, the copy of the template sequence may, in some embodiments, defer from the template sequence by the methylation status of the nucleobase sequence. For example, in some embodiments, the template sequence includes methylated and non-methylated cytosine bases, while substantially all of the cytosine bases in the copy of the template sequence are methylated. As further described herein, non-methylated cytosine bases may be converted to uracil bases. Accordingly, in a converted nucleic acid construct, the nucleobase sequence of the converted template sequence may differ from the copy of the template sequence (i.e., the original template sequence) based on a methylation pattern of the original template sequence. That is, cytosine bases protected by methylation in the template sequence will be retained as cytosine bases in the converted template sequence: in contrast, un-methylated cytosine bases in the template sequence will be uracils in the converted template sequence. In some embodiments, the nucleic acid molecule construct is a DNA construct. In some embodiments, the nucleic acid molecule construct is an RNA construct.

The nucleic acid construct strand (e.g., the nucleic acid molecule construct strand) may be configured such that the template sequence and the copy of the template sequence can be sequenced simultaneously. Accordingly, the nucleic acid construct strand can include two sequencing primer regions (i.e., an original sequencing primer region and a copy of the sequencing primer region). The sequencing primer region can be included 5′ of the template sequence, and a copy of the sequencing primer can be included 5′ of the copy of the template sequence (e.g., between the template sequence and the copy of the template sequence). That is, the original sequencing primer region is disposed 5′ to the template sequence on the nucleic acid molecule construct strand and the copy of the sequencing primer region is disposed 5′ of the copy of the template sequence in the nucleic acid molecule construct strand. The nucleic acid molecule construct strand may further include a nucleic acid linker separating the template sequence and the copy of the template sequence. Thus, the nucleic acid molecule construct strand can include, from 5′ to 3′, a first sequencing primer region, a first template sequence, a first nucleic acid linker, a copy of the first sequencing primer region, and a copy (e.g., a single copy) of the template sequence.

A nucleic acid construct, for example a construct that may be used in accordance with the methods described, herein can include a first nucleic acid construct strand and a second nucleic acid construct strand, which may hybridize to each other (e.g., in water at 25° C.). The first and second nucleic acid strands can be derived from a nucleic acid duplex, which may be obtained from a patient sample. The nucleic acid duplex is, in some embodiments, a DNA duplex. For example, the nucleic acid duplex may be a DNA fragment from a tissue sample or a cell-free DNA (cfDNA sample). In some embodiments, the nucleic acid duplex may be an RNA duplex, for example, the nucleic acid duplex may be an RNA fragment from a viral sample.

The first strand of the construct can correspond to the “top” strand of the nucleic acid duplex, and the second strand of the construct can correspond to the “bottom” strand of the nucleic acid duplex (where the top and bottom strands of the nucleic acid duplex may hybridize to each other, e.g., in water at 25° C.). The nucleic acid duplex can include a first template sequence in the top strand and a second template sequence in the bottom strand, and the template sequences are used to generate the nucleic acid construct. Similar to the first nucleic acid construct strand, the second strand of the nucleic acid construct can include two copies of the second template sequence, which may be identical copies or may differ based on the methylation profile of the second template sequence (for example, if used in a method to determine the methylation profile of the second template sequence).

Accordingly, a nucleic acid molecule can include a first construct strand and a second construct strand, wherein: the first construct strand comprises, from 5′ to 3′, a first sequencing primer region, a first template sequence, a first nucleic acid linker, a copy of the first sequencing primer region, and a copy (e.g., a single copy) of the first template sequence; and the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy (e.g., a single copy) of the second template sequence, and a copy of the second sequencing primer region.

The first template sequence and the copy of the first template sequence of the first strand in the nucleic acid construct may be separated by a first nucleic acid linker. Similarly, the second template sequence and the copy of the second template sequence of the second strand in the nucleic acid construct may be separated by a second nucleic acid linker. The first nucleic acid linker and the second nucleic acid linker may be reverse complements of each other. For example, the first nucleic acid linker and the second nucleic acid linker may be synthesized using the construct synthesis methods described herein.

The linker may be derived during synthesis of the nucleic acid construct, which can rely on an extension reaction performed on a partially circularized nucleic acid molecule as further described herein. The linker may be long enough to allow for an appropriate curvature of the partially circularized nucleic acid while still allowing a template sequence to function as a template during the extension reaction. For example, the linker may be long enough to form a circle or partial circle (e.g., in water at 25° C.) when hybridized to the nucleic acid duplex. In some implementations, the first nucleic acid linker and/or second nucleic acid linker is about 30 bases in length or more (e.g., about 40 bases in length or more, about 50) bases in length or more, about 60) bases in length or more, about 70 bases in length or more, about 80 bases in length or more, about 90 bases in length or more, or about 100 bases in length or more). The linker length may be set to a maximum length to avoid over-winding of the nucleic acid molecule. The maximum length of the linker may depend on the length of the template. For example, in some implementations, the first nucleic acid linker and/or second nucleic acid linker is about the length of the first template sequence or the second template sequence, or less. In some implementations, the first nucleic acid linker and/or second nucleic acid linker is between about 20% and about 100% (e.g., about 20% to about 30%, about 30% to about 40%, about 40% to about 50%, about 50% to about 60%, about 60% to about 70%, about 70% to about 80%, about 80% to about 90%, or about 90% to about 100%) of the length of the first template sequence or the second template sequence.

The nucleic acid construct may include a barcode sequence. The barcode sequence may include identification information, such as a unique molecular identifier (UMI), a sample barcode (also known as a “sample index”), or both. A nucleic acid strand of the construct may include a barcode sequence and a copy of the barcode sequence. For example, the nucleic acid strand can include a barcode sequence associated with the template sequence and a copy of the barcode associated with the copy portion. The construct may be configured such that the barcode sequence is sequenced with the template sequence (and, if present, the copy of the barcode sequence may be sequenced with the copy of the template sequence). For example, the barcode sequence may be positioned between the sequencing primer region and the template sequence. Thus, when sequencing is initiated (for example, by hybridization of a sequencing primer to the sequencing primer region), the barcode may be sequenced, followed by the template sequence, in a single read. Similarly, the copy of the barcode region may be positioned between the copy of the sequencing primer region and the copy portion. The second construct strand may include a reverse complement of the barcode sequence.

The nucleic acid molecule construct may optionally include a preamble sequence, and further optionally a copy of the preamble sequence. A preamble sequence is a relatively short sequence that includes at least one base type of each sequenced base (e.g., A. T. C, and G) so that the signal from each base type can be normalized during sequencing. Thus, the preamble sequence, if present, may be sequence along with the template sequence and/or copy of the template sequence. For example, the preamble sequence may be positioned between the sequencing primer region and the template sequence, for example adjacent to the barcode sequence. Similarly, the copy of the preamble sequencing may be positioned between the copy of the sequencing primer region and the copy of the template sequence. The second construct strand may include a reverse complement of the preamble sequence.

The first construct strand of the nucleic acid construct may further include forward and reverse amplification primer regions. Amplification primers can hybridize at the amplification primer regions (i.e., the sequence or reverse complement). The forward and reverse amplification regions may be positioned on opposite ends of the nucleic acid molecule construct strand, which allows for amplification (e.g., PCR amplification, such as emulsion PCR (ePCR) amplification) of the nucleic acid molecule construct. The second construct strand of the nucleic acid construct may include reverse complement sequences of the forward and reverse amplification primer regions.

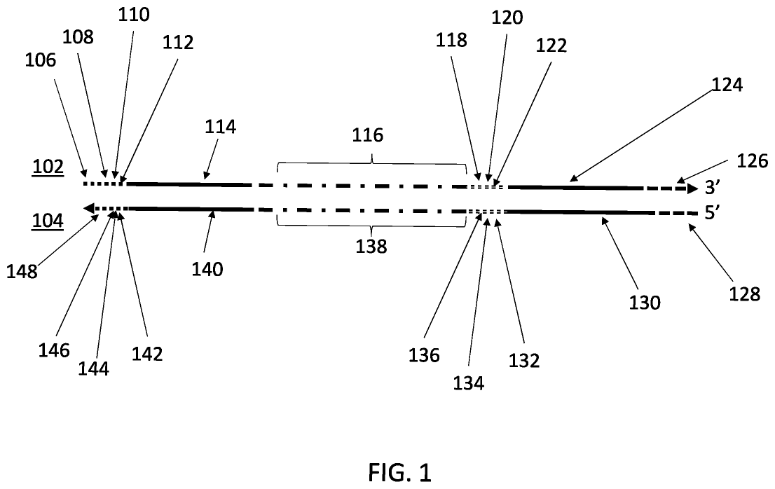

FIG. 1 illustrates an exemplary nucleic acid construct described herein. The construct includes a top strand (i.e., first construct strand) 102 and a bottom strand (i.e., second construct strand) 104. The first construct strand 102 includes, from 5′ to 3′, a forward amplification primer region 106, a first sequencing primer region 108, a first preamble sequence 110, a first barcode region 112 (which may include a UMI or sample barcode, or both in either order), a first template sequence 114, a linker sequence 116, a copy of the first sequencing primer region 118, a copy of the first preamble sequence 120, a copy of the first barcode region 122 (which may include a copy of the UMI or a copy of the sample barcode, or both, in the same order as present in first barcode region 112), a copy of the first template sequence 124, and a reverse amplification primer region 126. The second strand 104 of the exemplary nucleic acid construct includes, from 5′ to 3′, a reverse complement of the reverse amplification primer region 128, a second template sequence 130 (which is a reverse complement of the first template sequence 114), a second barcode region 132 (which is a reverse complement of the first barcode region 112), a second preamble sequence 134 (which is a reverse complement of the first preamble sequence 110), a second sequencing primer region 136 (which is a reverse complement of the first sequencing primer region 108), a second linker sequence 138 (which is a reverse complement of the first linker sequence 116), a copy of the first template sequence 140, a copy of the second barcode region 142, a copy of the second preamble sequence 144, a copy of the second sequencing primer region 146, and a reverse complement of the forward amplification primer region 148.

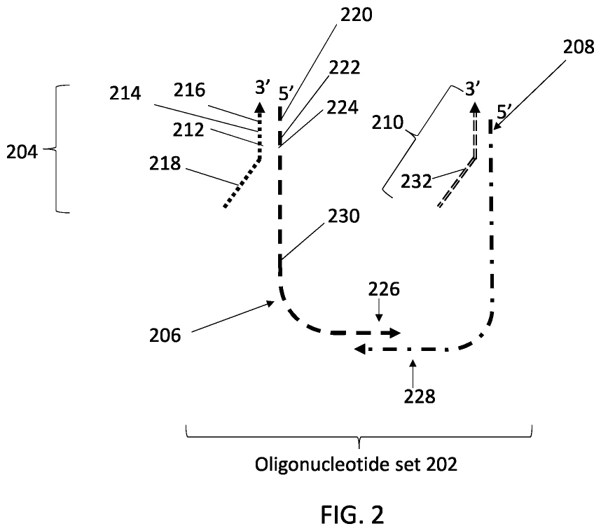

The nucleic acid construct may be synthesized using a concatenating synthesis process that includes the use of a set of oligonucleotides. The oligonucleotide set includes four oligonucleotides, portions of which hybridize (e.g., through reverse complementarity) to form a complex comprising the four-oligonucleotides. A portion of an oligonucleotide refers to a length of the nucleotide less than the total nucleotide length. The following discussions refer to a “3′ portion” and a “5′ portion” of the oligonucleotide. The appellation 3′ portion or 5′ portion (and 3′ end or 5′ end) is to indicate the proximal location of the referenced oligonucleotide portion, although the referenced portion need not be at the extreme 3′ terminus or 5′ terminus, respectively, of the oligonucleotide. In some implementations, the referenced 3′ portion or 5′ portion is within 10, 9, 8, 7, 6, 5, 4, 3, 2, or 1 bases of the respective 3′ terminus or the 5′ terminus: or the referenced 3′ portion of 5′ portion may be at the 3′ terminus or the 5′ terminus. As shown in FIG. 2, the oligonucleotide set 202 can assemble such that a 3′ portion of the first oligonucleotide 204 hybridizes to a 5′ portion of the second oligonucleotide 206, a 3′ portion of the second oligonucleotide 206 hybridizes to a 3′ portion of the third oligonucleotide 208, and a 5′ portion of the third oligonucleotide 208 hybridizes to a 3′ portion of the fourth oligonucleotide 210.

The 3′ portion of the first oligonucleotide 204 can include the first sequencing primer region 212. The sequence of the sequencing primer region may be, in some instances, the same sequence as a sequencing primer that is used to sequence the second template region or the copy of the first template region. The 3′ portion of the first oligonucleotide 204 may further include the first preamble sequence 214, the first barcode sequence 216, or both. The first preamble sequence 214 and/or the first barcode sequence 216, if present, may be proximal to the 3′ terminus of the first oligonucleotide 204 relative to the first sequencing primer region 212 (e.g., so that the first preamble sequence and the first barcode sequence will be sequenced during primer extension from a sequencing primer hybridized to the first sequencing primer region). The 5′ portion of the first oligonucleotide 204 may include a forward amplification primer region 218. The sequence of the forward amplification primer region 218 may, in some embodiments, be the same as a forward amplification primer used to amplify the nucleic acid construct or a derivative thereof (e.g., a converted nucleic acid construct).

The 5′ portion of the second oligonucleotide 206 can hybridize to 3′ portion of the first oligonucleotide 204 and can be a reverse complement of 3′ portion of the first oligonucleotide 204 (or substantially identical to a reverse complement of 3′ portion of the first oligonucleotide 204). The 5′ portion of the second oligonucleotide 206 can include a second barcode sequence 220, which may be a reverse complement of (and can hybridize to) the first barcode sequence 216. The 5′ portion of the second oligonucleotide 206 can include a second preamble sequence 222, which may be a reverse complement of (and can hybridize to) the first preamble sequence 214. The 5′ portion of the second oligonucleotide 206 can include a second sequencing primer region 224, which may include a sequence that is a reverse complement of (and can hybridize to) the first sequencing primer region 212.

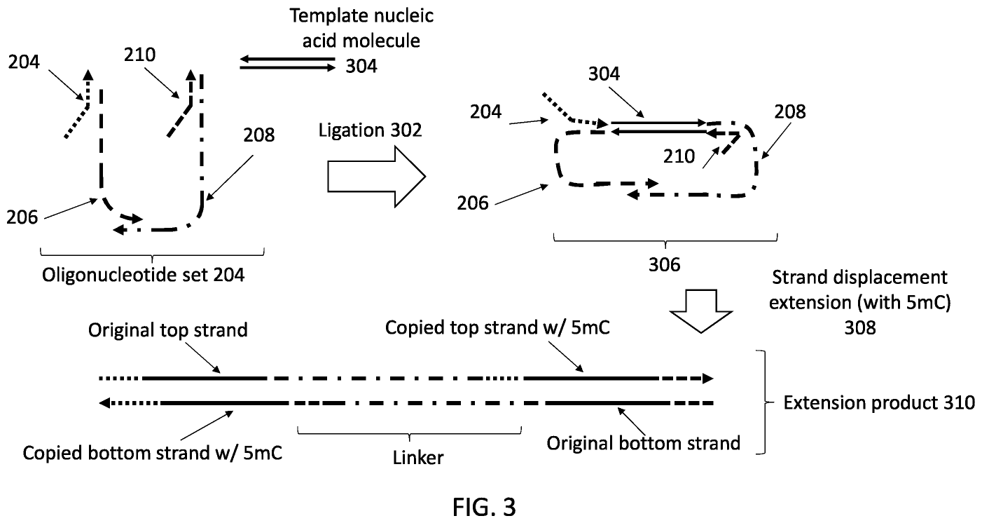

The 3′ portion 226 of the second oligonucleotide 206 can hybridize to 3′ portion 228 of the third oligonucleotide 208. A nucleic acid sequence 230 can separate 5′ portion of the second oligonucleotide 206 from 3′ portion of the second oligonucleotide 206. The 5′ portion of the third oligonucleotide 208 hybridizes to 3′ portion of the fourth oligonucleotide 210. The fourth oligonucleotide 210 can include a reverse amplification primer region. Preferentially, a reverse amplification primer region 232 is located in 5′ portion of the fourth oligonucleotide 210 (e.g., the region of fourth oligonucleotide 210 that does not hybridize to third oligonucleotide 208). This is advantageous in cases where the reverse amplification primer region is used for amplifying the nucleic acid construct prior to sequencing since 3′ region of fourth oligonucleotide is duplicated during construction of the nucleic acid molecule: the presence of multiple reverse amplification primer regions will result in a mixture of products from an amplification step (e.g., copies of the entire nucleic acid construct and copies of only the original top portion of the nucleic acid construct (see FIG. 3).

Optionally, the second oligonucleotide is cross-linked to the third oligonucleotide through a crosslinker, which may be a reversible crosslinker. Exemplary reversible crosslinkers include a psoralen crosslinker or a 3-cyanovinylcarbazole (CNVK) crosslinker. Other reversible crosslinkers are known in the art. The crosslinker can crosslink the portion of the second oligonucleotide that hybridizes to the portion of the third oligonucleotide. For example, 3′ portion of the second oligonucleotide can include a first member of a crosslinker (e.g., a reversible crosslinker) and 3′ portion of the third oligonucleotide can include a second member of the crosslinker.

The nucleic acid construct can then be produced by coupling the oligonucleotide set to a template molecule and performing an extension reaction. As illustrated by the exemplary method illustrated in in FIG. 3, oligonucleotide set 204 (e.g., as described in FIG. 2) may be ligated 302 to the template nucleic acid 304. For example, a 3′ terminus of the first oligonucleotide can be ligated to a 5′ terminus of the first strand of the template nucleic acid, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand.

In some implementations, the second oligonucleotide is cross-linked to the third oligonucleotide prior to the ligating (e.g., to prevent decoupling of strands 206 and 208 in oligonucleotide set 204). In some implementations, the second oligonucleotide is cross-linked to the third oligonucleotide after the ligating. The resulting construct is a partially circular nucleic acid molecule 306 that includes a first strand comprising a first template sequence and a second strand comprising a second template sequence, wherein the first template sequence is a reverse complement of the second template sequence.

An extension reaction 308 is then performed on the partially circular nucleic acid molecule. The 3′ terminus of the second oligonucleotide is extended using, in order, a portion of the third oligonucleotide, the first strand and the first oligonucleotide as a template. The 3′ terminus of the third oligonucleotide is also extended using, in order, a portion of the second oligonucleotide, the second strand, and the fourth oligonucleotide as a template. The extension reaction can occur in the presence of a nucleotide regent that includes nucleotides necessary for the extension reaction (e.g., A. T. C, and G bases). In some implementations, strand displacement may occur in the presence of 5-methylcytisine (e.g., preparatory to a deamination reaction and sequencing to determine methylation status) (shown in FIG. 3). In some implementations, strand displacement may be performed with unmethylated cytosine (e.g., in cases where no subsequent methylation status sequencing is desired). In some implementations, the optional reversible cross-linker is reversed after the strand extension reactions. The resulting construct 310 includes a first strand comprising the first template sequence portion (“original top”) and a first copy portion (“copied top”), and a second strand comprising the second template sequence portion (“original bottom”) and a second copy portion (“copied bottom”). The resulting extension product 310 is the nucleic acid molecule construct provided herein, such as the exemplary nucleic acid molecule construct shown in FIG. 1.

The template nucleic acid may be, for example, a duplex nucleic acid molecule obtained from the biological sample from a subject. In such cases, the template nucleic acid molecule includes a first strand comprising a first template sequence and a second strand comprising a second template sequence. The template nucleic acid may have a naturally occurring methylation profile. The template nucleic acid may be prepared for construct synthesis, for example by nucleic acid end repair and/or A-tailing. The first strand and/or second strand of the nucleic acid molecule may be a cfDNA molecule. The template nucleic acid molecule may be, in some embodiments, up to 100 bases (bp), 150 bp, 200 bp, 250 bp, 300 bp, 400 bp, 500 bp, 600 bp, 700 bp, 800 bp, 900 bp or 1,000 bp in length. In some embodiments, the length can be longer than 1,000 bp such as up to 1.1 kilobases (kb), 1.2 kb, 1.3 kb, 1.4 kb, 1.5 kb, 1.6 kb, 1.7 kb, 1.8 kb, 1.9 kb, or 2 kb, or longer.

The template nucleic acid molecule(s) used in the methods described herein may be obtained from any suitable biological source, for example a tissue sample, a blood sample, a serum sample, a cerebrospinal fluid sample, a plasma sample, a saliva sample, a fecal sample, or a urine sample. In some embodiments, RNA polynucleotides are reverse transcribed into DNA polynucleotides to be used as template nucleic acid molecules. In some embodiments, the template nucleic acid molecule is a cell-free DNA (cfDNA), such as a circulating tumor DNA (ctDNA) or a fetal cell-free DNA.

In some instances, the nucleic acid constructs described herein may be amplified prior to sequencing (e.g., PCR, ePCR, etc.). Amplification may occur in the presence of canonical deoxynucleotides (e.g., A, C, T, and G, excluding methylated cytosine), which cause uracil in the converted nucleic acid construct to be replaced with thymine in the resulting amplicons. In some instances, the nucleic acid constructs described herein may be sequenced without any amplification (e.g., single molecule sequencing). The nucleic acid constructs described herein may be sequenced, for example to determine a sequence of the first template sequence and/or a sequence of the second template sequence. Because the construct may be designed for sequencing of the converted template sequence and the copy of the template sequence simultaneously (e.g., by including both a sequencing primer region and a copy of the sequencing primer region in the construct), a differential between sequencing signals (e.g., between a signal originating from the converted template sequence and a signal originating from the copy of the template sequence) can be detected, which differential can indicate the presence of sequencing errors and/or sequence differences between the original template sequence and the copy of the template sequence.

### Nucleic Acid Constructs for Methylation Profiling

The method of making the nucleic acid molecule construct described herein may be modified, in some embodiments, to make a construct suitable for methylation profiling. For example, the method may be modified by performing the extension reaction in the presence of methylated cytosine (e.g., 5-methylcytotsine). In some implementations, the extension reactions occur in the presence of a nucleotide reagent that includes methylated cytosine bases (e.g., 5-methylcytosine). For example, substantially all cytosine bases in the nucleotide reagent may be methylated cytosine bases. For example, a method of making the nucleic acid construct can include performing extension reactions, in the presence of methylated cytosine (e.g., wherein substantially all or all cytosine bases present in the extension are methylated cytosine (e.g., 5-methycytosistine), on a partially circular nucleic acid molecule comprising a first strand comprising a first template sequence and a second strand comprising a second template sequence, wherein the first template sequence is a reverse complement of the second template sequence. The method thereby generates a nucleic acid molecule that includes a first strand comprising from 5′ to 3′, a first sequencing primer region, a first template sequence, a first nucleic acid linker, a copy of the first sequencing primer region, wherein substantially all cytosine bases in the copy of the first sequencing primer region are methylated, and a copy of the first template sequence, wherein substantially all cytosine bases in the copy of the first template sequence are methylated; and a second construct strand comprising, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, wherein substantially all cytosine bases in the copy of the second template sequence are methylated, and a copy of the second sequencing primer region, wherein substantially all cytosine bases in the copy of the second sequencing primer region are methylated.

The nucleic acid construct may be synthesized in the presence of nucleotides (e.g., deoxynucleotides) that include 5-methylcytosine (5mC) in place of canonical cytosine (e.g., A. T. G, and 5mC, and excluding C) such that the resulting construct includes a first template sequence (with the original methylation profile) and a copy of the first template sequence, wherein substantially all cytosine bases in the copy of the first template sequence are methylated (e.g., 5-methylcytosine); and a second strand comprising a second template sequence (with the original methylation profile) and a copy of the second portion, wherein substantially all cytosine bases in the second copy portion are methylated (e.g., 5-methylcytosine). The first and second template sequences may therefore include methylated cytosine (i.e., naturally occurring methylated cytosine) and non-methylated cytosine (i.e., naturally occurring non-methylated cytosine), while substantially all (or all) cytosine bases in the copies of the first and second template sequences are methylated cytosine (i.e., 5-methylcytosine). The nucleic acid construct can be subjected to a conversion reaction, wherein non-methylated cytosine is converted to uracil. If the first (or second) copy includes only methylated cytosine, the sequence of the first (or second) copy is not modified and remains identical to the original first (or second) template sequence. The first (or second) template sequence (i.e., a template sequence in the original template nucleic acid molecule), however, may include both methylated and non-methylated cytosine, so the conversion reaction will alter the sequence of the first (or second) template sequence such that substantially all of the non-methylated cytosine bases in the first (or second) template sequence become uracil bases. “Substantially all” in this context indicates that the conversion reaction may be incomplete such that a small portion (e.g., less than 10%) of non-methylated cytosine may remain as non-methylated cytosine bases. In some cases, subsequent to the conversion reaction, at most about 10.0%, 9.5%, 9.0%, 8.5%, 8.0%, 7.5%, 7.0%, 6.5%, 6.0%, 5.5%, 5.0%, 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.1%, or less of non-methylated cytosine bases in the first (or second) template sequence remain as non-methylated cytosine bases. The nucleic acid construct resulting from the conversion reaction thus comprises a first strand and a second strand. The first strand comprises a first converted template sequence (corresponding to the template sequence post-conversion reaction) and a copy of the first template sequence, wherein substantially all cytosine bases in the copy of the first template are methylated cytosine, and substantially all bases in the first template sequence that correspond to cytosine bases in the copy of the first template sequence are methylated cytosine or uracil (depending on the methylation status of the original first template sequence). The second strand comprises a second converted template sequence and a copy of the second template sequence, wherein substantially all cytosine bases in the copy of the second template sequence are methylated cytosine, and substantially all bases in the converted second template sequence that correspond to cytosine bases in the copy of the second template sequence are methylated cytosine or uracil (depending on the methylation status of the original second template sequence).

The nucleic acid construct resulting from the conversion reaction may be amplified (e.g., through PCR amplification) in the presence of canonical deoxynucleotides (A, G, C, T), which amplification replaces any uracil bases with thymine bases. Thus, the amplified nucleic acid construct comprises a first strand comprising a first converted template sequence and a copy of the first template sequence (wherein the cytosine bases may or may not be methylated depending on the methylation status of cytosine in the amplification reagent), and substantially all bases in the converted first template sequence that correspond to cytosine bases in the copy of the first template sequence are cytosine or thymine (depending on the methylation status of the cytosine bases in the original first template sequence); and a second strand comprising a converted second template sequence and a copy of the second template sequence portion, and substantially all bases in the converted second template sequence that correspond to cytosine bases in the copy of the second template sequence are cytosine or thymine (depending on the methylation status of the original second template sequence).

Alternatively, the construct may be synthesized in the presence of only canonical nucleotides (e.g., deoxynucleotides) (e.g., A. T. C, and G, with no methylated cytosine nucleotides used to synthesize the construct). In such cases, the resulting construct includes a first strand comprising a first template sequence and a copy of the first template sequence, wherein substantially all (or all) cytosine bases in the copy of the first template sequence are non-methylated; and a second strand comprising a second template sequence and a copy of the second template sequence, wherein substantially all (or all) cytosine bases in the copy of the second template sequence are non-methylated. The construct may be subjected to a conversion reaction wherein methylated cytosine is converted to uracil, which provides a converted nucleic acid construct that includes a first strand comprising a converted first template sequence and a copy of the first template sequence, wherein at least a portion of bases in the converted first template sequence that correspond to cytosine bases in the copy of the first template sequence portion are uracil; and a second strand comprising a converted second template sequence and a copy of the second template sequence, wherein at least a portion of bases in the converted second template sequence that correspond to cytosine bases in the copy of the second template sequence are uracil. Cytosine bases in the first (or second) copy portion remain as cytosines when the construct is synthesized using non-methylated cytosine. The nucleic acid construct may be amplified (e.g., through PCR amplification) in the presence of canonical deoxynucleotides (A. G. C. T), which replaces the uracil bases with thymine bases. Thus, the amplified nucleic acid construct includes a first strand comprising a converted first template sequence and a copy of the first template sequence, wherein at least a portion of bases in the converted first template sequence that correspond to cytosine bases in the copy of the first template sequence are thymine; and a second strand comprising a converted second template sequence and a copy of the second template sequence, wherein at least a portion of bases in the converted second template sequence that correspond to cytosine bases in the copy of the second template sequence are thymine. Cytosine bases in the first (or second) template sequence that were not methylated in the original first (or second) template are not converted, and thus remain cytosine.

When the copies of the first or second template sequences are synthesized using methylated cytosine (i.e., omitting non-methylated cytosine) and non-methylated cytosine is converted to uracil or thymine, or alternatively when the first or second copy portions are synthesized using non-methylated cytosine (i.e., omitting methylated cytosine) and methylated cytosine is converted to uracil or thymine, the copies of the first and second template sequences retain the sequence of the first and second template sequences, respectively. Thus, when the first and second template sequences are reverse complements of each other (for example, when they are a nucleic acid duplex from a biological sample of a subject), the copy of the first template sequence is a reverse complement of the copy of the second template sequence.

Once the nucleic acid construct is generated through the extension reaction, non-methylated cytosine in the construct may be converted to uracil. Conversion may be chemical or enzymatic. For example, in some embodiments, the nucleic acid construct is treated with bisulfite to convert non-methylated cytosine to uracil. Alternatively, an enzymatic method may be used, for example by treating the nucleic acid construct with an enzyme that converts non-methylated cytosine to uracil, for example using NEBNext® Enzymatic Methyl-seq Kit (New England BioLabs), a ten-eleven translocation methylcytosine dioxygenase 2 (TET2) enzyme, or an APOBEC2 enzyme. Alternatively, methylated cytosine in the construct may be converted to uracil. See, for example, Liu et al., Bisulfate-free direct detection of 5-methylcytosine and 5-hydroxymethylcytosine at base resolution, Nature Biotechnology, vol. 37, pp. 424-429 (2019). This process of converting non-methylated cytosine to uracil (or, alternatively, methylated cytosine to uracil) results in a converted nucleic acid molecule comprising a first converted strand comprising a converted first template sequence and a copy of the first template sequence, or a second converted strand comprising a converted second template sequence and a copy of the second template sequence.

The converted nucleic acid molecule may be amplified. Amplification may occur in the presence of canonical deoxynucleotides (e.g., A, C, T, and G, excluding methylated cytosine), which cause uracil in the converted nucleic acid construct to be replaced with thymine in the resulting amplicons. The resulting construct includes a converted first template sequence (corresponding to the original first template sequence) and a copy of the first (original) template sequence, wherein the converted first template sequence and the copy of the first template sequence differ based on the methylation profile of the original first template sequence. The construct also includes a converted second template sequence (corresponding to the original second template sequence) and a copy of the (original) second template sequence, wherein the converted second template sequence and the copy of the second template sequence differ based on the methylation profile of the (original) second template sequence.

The nucleic acid constructs described herein may be sequenced, for example to determine a methylation profile of the first template sequence and/or a methylation profile of the second template sequence. That is, the difference between the sequence of the first portion and the first copy portion can indicate the methylation profile of the first template sequence, and the difference between the sequence of the second portion and the second copy portion can indicate the methylation profile of the second template sequence. Because the construct may be designed to sequence the converted template sequence and the copy of the template sequence simultaneously (by including a sequencing primer region and a copy of the sequencing primer region in the construct), a differential between sequencing signals can be determined, which indicates the methylation pattern of the original template sequence.

### Targeted Capture

Capture probes may be used to enrich targeted sequences (e.g., targeted CpG sequences) prior to sequencing. Pools of sequencing constructs formed from template nucleic acid molecules, for example, those obtained from a sample from a subject, may include many template sequence of low interest (for example, templates sequences that include no CpG methylation sites, or are otherwise from a region of the genome that is of low interest). Thus, sequencing all template sequences in the pool can result in unnecessary sequencing throughput, which uses additional reagents and analytical power to interpret the sequencing data.

To enrich for targeted sequences, a pool of converted constructs (e.g., after completing a non-methylated cytosine to uracil conversion reaction, or after an amplification reaction to convert uracil to thymine residues) can be contacted with a plurality of capture probes. The capture probes can include a capture sequence (i.e., a nucleotide sequence) configured to target a region (e.g., CpG site) in the original template sequence (i.e., prior to conversion). The targeted region may be a predetermined CpG site, for example a CpG site from within a selected gene. The capture sequence may be, for example, at least 10 bases in length, at least 20 bases in length, at least 30 bases in length, at least 40 bases in length, at least 50 bases in length, at least 60 bases in length, at least 70 bases in length, at least 80 bases in length, at least 90 bases in length, at least 100 bases in length or longer. In addition to the capture sequence, the capture probe may optionally include a 5′ and/or 3′ flanking region, which does not hybridize to the targeted sequence. The capture probe may also include an binding moiety (e.g., biotin), which can be used to separate nucleic acid molecules hybridized to the capture probe from those that do not hybridize to the capture probe.

The capture probes may be mixed with the pool of nucleic acid molecule constructs after amplification of the nucleic acid molecule constructs. This can help ensure that sufficient nucleic acid material is available for efficient capture.

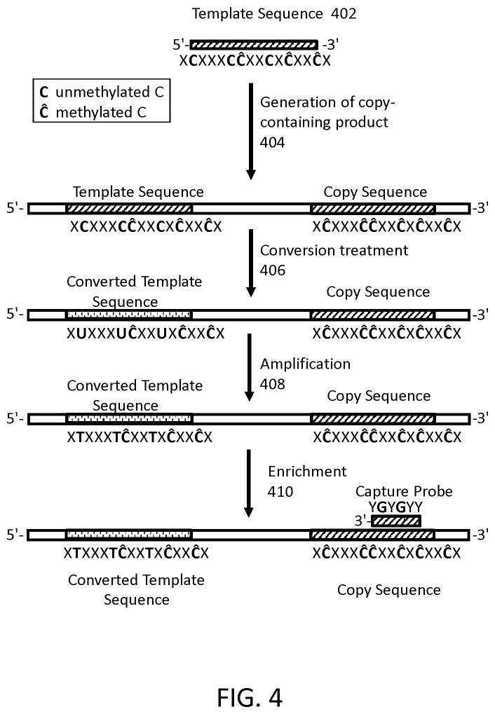

FIG. 4 shows an exemplary method for targeted enrichment of a CpG site according to some embodiments. At 402, a template nucleic acid molecule is provided, which includes a template sequence. The template sequence may include one or more CpG sites and/or include one or more methylated cytosine residues. The template sequence may include one or more unmethylated cytosine residues. The template nucleic acid molecule may be a duplex nucleic acid molecule and can include a second template sequence that is a reverse complement of the first template sequence. At 404, a nucleic acid molecule construct is generated, which includes the template sequence and a copy of the template sequence (i.e., a “copy sequence”), which sequences differ only in the methylation status of the cytosine residues. For example, the nucleic acid molecule construct may be generated in the presence of a nucleotide reagent that includes methylated cytosine bases (e.g., all or substantially all cytosine bases in the nucleotide reagent are methylated) such that when the nucleic acid molecule construct is generated, the cytosine residues in the copy sequence are all methylated or substantially all cytosine residues in the copy sequence are methylated.

The nucleic acid molecule construct formed at 404 may be made according to the methods described herein. For example, the template nucleic acid molecule may be combined with an oligonucleotide set comprising a first oligonucleotide, a second oligonucleotide, a third oligonucleotide, and a fourth oligonucleotide. A 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide. The oligonucleotide set may then be ligated to the template nucleic acid molecule. For example, a 3′ terminus of the first oligonucleotide can be ligated to a 5′ terminus of the first strand, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand. The ligation reaction thereby forms a partially circular nucleic acid molecule. After ligation, extension reactions can be performed in the presence of the nucleotide reagent that includes methylated cytosine bases to form the nucleic acid molecule construct.

At 406, unmethylated cytosine residues in the nucleic acid molecule are converted to uracil residues. This generates a converted nucleic acid molecule that includes the copy sequence (which is the same as the original template sequence, as cytosine bases in the copy sequence were methylated and therefore protected from the conversion reaction) and a converted template sequence, which includes cytosine bases (corresponding to methylated cytosine bases in the original template strand) and uracil bases (corresponding to unmethylated cytosine bases in the original template strand). The conversion reaction may be performed, for example, according to the methods described herein.

The converted nucleic acid construct may be amplified (e.g., through PCR amplification) in the presence of canonical deoxynucleotides (A, G, C, T) at 408. Amplification replaces any uracil bases with thymine bases in the resulting amplicon. Thus, the amplicons include a converted template sequence that includes cytosine nucleotides (corresponding to methylated cytosine nucleotides in the original template sequence) and thymine nucleotides (corresponding to unmethylated cytosine nucleotides and original thymine nucleotides in the original template sequence).

At 410, targeted template sequences are enriched. A capture probe configured to hybridize to at least a portion of the copy sequence is contacted with the amplicon, thus allowing the capture probe to hybridize to the amplicon. In some implementations, the capture probe may be contacted with the converted nucleic acid molecule, for example prior to amplification or in a method that does not include an amplification step. Because the converted template sequence differs from the copy sequence based on methylation status and conversion, the capture probe binds the copy sequence.

The capture probe may be designed such that it is agnostic to the original methylation status as a copy of the original sequence (prior to conversion) is conserved post-conversion. That is, the capture probe may be designed to capture pre-conversion sequences in the template sequence. Beneficially, such methods may achieve enrichment of targeted regions that is unbiased as to the methylation status estimated in the design of the capture probe. This is advantageous to methods where the nucleic acid population to be enriched, post-conversion and amplification, does not include a copy of the original sequence (pre-conversion) and thus capture probes have to be designed to capture a target region based on an estimated methylation status of the target region, or a given composition of probes have to be designed to capture various degrees of methylation status of the target region.

After hybridization, the hybridized duplex (i.e., the complex that includes the capture probe and amplicon (or converted nucleic acid molecule) can be separated from nucleic acid molecules that do not hybridize to a capture probe.

The method may be used to isolate targeted template sequences from a pool. Thus, in some embodiments, the method may include providing a plurality of nucleic acid molecules, each comprising, in the same strand, a template sequence and a copy sequence, wherein the copy sequence is a copy of the template sequence except that substantially all cytosine bases in the copy sequence are methylated, wherein a first portion of nucleic acid molecules in the plurality of nucleic acid molecules comprises a different template sequence than a second portion of nucleic acid molecules in the plurality of nucleic acid molecules: converting unmethylated cytosine residues in the plurality of nucleic acid molecules to uracil residues, thereby generating a plurality of converted nucleic acid molecules, each converted nucleic acid molecule comprising the copy sequence and a converted template sequence; and hybridizing a plurality of capture probes to at least a portion of the copy sequence. The method may further include amplifying the plurality of converted nucleic acid molecules, thereby substituting uracil residues in the converted template sequence with thymine residues to form a plurality of amplicon, wherein the capture probes hybridize to at least a portion of the copy sequence in the amplicons.

Once separated from non-targeted regions, the nucleic acid molecules may be sequenced as described herein. For example, the nucleic acid molecules may be sequenced to determine a methylation profile of the template sequence.

### Flow Sequencing Methods

Sequencing data can be generated using a flow sequencing method that includes extending a primer bound to a template polynucleotide molecule according to a predetermined flow cycle where, in any given flow position, a set of nucleotide base types (e.g., 1, 2, 3, or 4 different base types selected from A, C, T and G) is accessible to the extending primer. In some instances, fewer base types provided in a given flow provide higher certainty about the precise nucleic acid sequence of the targeted template but provides a smaller sequencing distance per flow. In some embodiments, at least some of the nucleotides of the particular type include a label, which upon incorporation of the labeled nucleotides into the extending primer renders a detectable signal. The resulting sequence by which such nucleotides are incorporated into the extended primer should be the reverse complement of the sequence of the template polynucleotide molecule. In some embodiments, for example, sequencing data is generated using a flow sequencing method that includes extending a primer using labeled nucleotides and detecting the presence or absence of a labeled nucleotide incorporated into the extending primer. Flow sequencing methods may also be referred to as “natural sequencing-by-synthesis.” or “non-terminated sequencing-by-synthesis” methods. Exemplary methods are described in U.S. Pat. No. 8,772,473; International Publication Number WO 2020/227143 A1; and International Publication Number WO 2020/0227137 A1; each of which is incorporated herein by reference in its entirety. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A. C. G. T. U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added) during a single flow step, although two, three, or four different types of nucleotides may be simultaneously introduced (e.g., in a single flow step) in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may be further divided into cycles (e.g., flow cycles). Nucleotides are added stepwise (e.g., in flow steps), which allows incorporation of the added nucleotides to the end of the sequencing primer of a complementary base in the template strand is present. The cycles may have the same order of nucleotides and number of different base types or a different order of nucleotides and/or a different number of different base types. However, no set of bases (i.e., the one or more different bases simultaneously used in a single flow step) corresponding to a given flow step is repeated in the same cycle as the term is used herein, which can provide as a marker to distinguish between different cycles. Solely by way of example, the order of a first cycle may be A-T-G-C and the order of a second cycle may be A-T-C-G. Further, one or more cycles may omit one or more nucleotides. Solely by way of example, the flow order of a first cycle may be A-T-G-C and the order of a second cycle may be A-T-C. Alternative orders may be readily contemplated by one skilled in the art. Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E, coli DNA polymerase I. T7 DNA polymerase, bacteriophage T4 DNA polymerase Φ29 (phi29) DNA polymerase, Taq polymerase, Tth polymerase. Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiment, the nucleotides introduced include only unlabeled nucleotides, and in some embodiments the nucleotides introduced include a mixture of labeled and unlabeled nucleotides. For example, in some embodiments, the proportion of labeled nucleotides compared to total nucleotides (e.g., the total introduced nucleotides) is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the proportion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the proportion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

In some embodiments, a combination of nucleotide types is introduced in one or more flow cycle steps during the course of primer extension (e.g., non-discrete addition of nucleotide types). For example, two different base types, such as G and C, may be used simultaneously in the same flow step. The addition of these two bases will permit primer extension if any complementary C and/or G bases are present. This accelerates extension of the primer by incorporating consecutive bases into the primer even if those bases are of different base types. In some embodiments, at least one step of the flow order includes 2 different base types. In some embodiments, at least one step of the flow order includes 3 different base types. In some embodiments, at least one step of the flow order includes 4 different base types. By way of example, in a flow step including two different base types, a first base type is labeled (e.g., in a proportion, as described above, of labeled nucleotides of first base type to total nucleotides of first base type) and a second base type is not labeled. In an additional example, in a flow step including three different base types, a first base type is labeled (e.g., in a proportion, as described above, of labeled nucleotides of first base type to total nucleotides of first base type), a second base type is not labeled, and a third base type is not labeled.

Prior to generating the sequencing data, the polynucleotide is hybridized to a sequencing primer to generate a hybridized template. The polynucleotide may be ligated to an adapter during sequencing library preparation. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Patent Ser. No. 10,344,328, which is incorporated herein by reference in its entirety.

Sequencing data, such as a flowgram, can be generated based on the detection of an incorporated nucleotide and the order of nucleotide introduction. Take, for example, the template sequences: CTG and CAG, and a repeating flow cycle of T-A-C-G (that is, sequential addition of T, A, C, and G nucleotides, which would be incorporated into the primer only if a complementary base is present in the template polynucleotide). An exemplary resulting flowgram is shown in Table 1, where 1 indicates incorporation of an introduced nucleotide, 0 indicates no incorporation of an introduced nucleotide, and an integer x>1 indicates incorporation of x introduced nucleotides. The flowgram can be used to determine the sequence of each respective template strand.

TABLE 1
Signals Detected from Example Sequences

| Flow Cycle | 1 | | | | 2 | | | |
|---|---|---|---|---|---|---|---|---|
| Flow Step | 1 | 2 | 3 | 4 | 1 | 2 | 3 | 4 |
| Flow Base | T | A | C | G | T | A | C | G |
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

A flowgram may be binary or non-binary. A binary flowgram detects the presence (1) or absence (0) of an incorporated nucleotide. A non-binary flowgram can more quantitatively determine a number of incorporated nucleotide from each stepwise introduction. For example, a sequence of CCG would incorporate two G bases, and any signal emitted by the labeled base would have a greater intensity as the incorporation of a single base. This is shown in Table 1. Thus, a non-binary flowgram also indicates the presence or absence of the base, and a non-binary flowgram can provide additional information including the number of bases incorporated at the given step.

Prior to generating the sequencing data, the polynucleotide is hybridized to a sequencing primer (e.g., at each sequencing primer region) to generate a hybridized template. The polynucleotide may be ligated to an adapter during sequencing library preparation. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Patent Ser. No. 10,344,328, which is incorporated herein by reference in its entirety.

Primer extension using flow sequencing allows for long-range sequencing on the order of hundreds or even thousands of bases in length. The number of flow steps or cycles can be increased or decreased to obtain the desired sequencing length. Extension of sequencing primers in the template sequence and in the copy of the template sequence can include one or more flow steps for stepwise extension of the primers using nucleotides having one or more different base types. In some embodiments, extension of the primers in the template sequence and in the copy of the template sequence includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into each of the primers in the template sequence and in the copy of the template sequence depends on the sequence of the template sequence and in the copy of the template sequence, and the flow order used to extend the primers. In some instances, the template sequence and the copy of the template sequence are each about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250) bases in length, about 250) bases to about 500 bases in length, about 500 bases to about 1000 bases in length, about 1000 bases to about 2000 bases in length, or about 2000 bases to about 4000 bases in length.

Beneficially, as the template sequence and the copy of the template sequence are obtained concurrently, there is no requirement to extend a primer molecule through the linker region of a nucleic acid construct. That is, fewer sequencing steps will be required than if a single sequencing primer were used to extend through the template sequence, through the linker region, and then through the copy of the template sequence.

When two sequences (e.g., a template sequence and a copy of the template sequence) are simultaneously sequenced, for example using the nucleic acid molecule construct described herein, signal intensity can be used to indicate a sequence differential between the two sequences. For example, two identical sequences should produce a signal that is approximately twice as intense as a single sequence. If the signal intensity for a particular flow drops or increases from than the 2-fold expected intensity, then the presence of a difference between the two sequences can be identified. In some cases, specific variations between the template sequence and the copy of the template sequence may be identified (i.e., by examination of the associated non-binary flowgram).

The methods described herein can include generating differential profile data that includes first sequencing data, comprising a nucleic acid sequence corresponding to a template sequence; and second sequencing data, comprising a nucleic acid sequence corresponding to the copy of the template sequence or the copy of the second template sequence. The first sequencing data and the second sequencing data may be generated simultaneously. For example, the construct may include a sequencing primer region associated with the template sequence, and a copy of the sequencing primer region associated with the copy of the template sequence. Sequencing primers combined with the construct can simultaneously hybridize to the sequencing primer region and the copy of the sequencing primer region, and the sequencing primers may be extended simultaneously, or substantially simultaneously, during sequencing. Differential profile data may be integrated data that includes the first and second sequencing data (i.e., a single signal at any given flow step that is the sum of the first and second sequencing data).

By way of example, consider a construct with a template sequence ATTGACCC (SEQ ID NO: 1) and an identical copy sequence. The resulting signal from simultaneous sequencing of the template and the copy sequences would be twice as intense at each flow step as compared with the signal that would be detected if only a single sequence (e.g., either the template or the copy) were sequenced. An exemplary non-binary flowgram indicating signal intensity at each flow step (assuming a repeated A-T-G-C flow order) is provided in Table 2. A deletion in the copy of the template sequence (e.g., ATGACCC, SEQ ID NO: 2) relative to the template sequence (SEQ ID NO: 1) would result in an altered signal intensity at the corresponding flow. See Table 2, flow cycle 1, flow step 2. An insertion in the copy of the template sequence (e.g., ATTTGACCC, SEQ ID NO: 3) would result in a different altered signal at the corresponding flow. See Table 2, flow cycle 1, flow step 2. Further, a substitution in the copy of the template sequence (e.g., ATAGACCC, SEQ ID NO: 4) would result in yet a different altered signal. See Table 2, flow cycle 1, flow step 2 onwards.

TABLE 2
Example of Expected Signals Detected from Simultaneous
Sequencing of Template and Copy Sequences,
Detection of Sequence Variation

| | Flow Cycle | | | | | | | | | | | |
|---|---|---|---|---|---|---|---|---|---|---|---|---|
| | 1 | | | | 2 | | | | 3 | | | |
| | Flow Step | | | | | | | | | | | |
| | 1 | 2 | 3 | 4 | 1 | 2 | 3 | 4 | 1 | 2 | 3 | 4 |
| | Flow Base | | | | | | | | | | | |
| | A | T | G | C | A | T | G | C | A | T | G | C |
| Identical | 2 | 4 | 2 | 0 | 2 | 0 | 0 | 6 | 0 | 0 | 0 | 0 |
| Template and | | | | | | | | | | | | |
| Copy | | | | | | | | | | | | |
| Deletion in | 2 | 3 | 2 | 0 | 2 | 0 | 0 | 6 | 0 | 0 | 0 | 0 |
| Copy | | | | | | | | | | | | |
| Insertion in | 2 | 5 | 2 | 0 | 2 | 0 | 0 | 6 | 0 | 0 | 0 | 0 |
| Copy | | | | | | | | | | | | |
| Substitution | 2 | 3 | 1 | 0 | 2 | 0 | 1 | 3 | 1 | 0 | 0 | 3 |
| in Copy | | | | | | | | | | | | |

The differential profile can be used to identify a difference between the template sequence and the copy of the template sequence. If the template sequence and the copy sequence were identical, the normalized signal intensity would be an integer value (i.e., the non-normalized signal intensity is an even integer or an approximately even value), depending on the number of contiguous identical bases (with larger homopolymers generating an increased normalized signal proportional to the number of identical bases in the homopolymer). A difference between the template sequence and the copy, however, would result in a non-integer normalized intensity at one or more flow positions.

The differential profile can be used to identify a difference between the template sequence and the copy of the template sequence. If the template sequence and the copy sequence were identical, the normalized signal intensity would be an integer value (i.e., the non-normalized signal intensity is an even integer or an approximately even value), depending on the number of contiguous identical bases (with larger homopolymers generating an increased normalized signal proportional to the number of identical bases in the homopolymer). A difference between the template sequence and the copy, however, would result in a non-integer normalized intensity at one or more flow positions.

The differential profile data may be used for quality control of sequencing reads in sequencing library data. During PCR amplification or sequencing, errors may be introduced that give rise to inaccurate or poor quality sequencing reads. If the template sequence and the copy of the template sequence were intended to be identical, but a differential was found in the differential profile data, the error-causing read may be filtered (i.e., removed) from the library sequencing data. Accordingly, in some embodiments, the method further comprises filtering library sequencing data to remove sequencing reads associated with a difference between the first sequencing data (comprising a nucleic acid sequence corresponding to the template sequence) and the second sequencing data (comprising a nucleic acid sequence corresponding to the copy of the template sequence). That is, the library sequencing data may be filtered by identifying sequencing reads where there are differences between the first sequencing data and the second sequencing data (e.g., to remove or tag sequencing reads with differences for purposes of downstream analysis).

### Methylation Status Data

As discussed above, the nucleic acid sequence of the copy of the template sequence is not altered by conversion of the methylated or non-methylated cytosine to uracil (and, after amplification, thymine). However, since the template sequence may retain its original methylation status (e.g., as obtained from the biological sample), in some cases, a subset of the cytosine bases in the template sequence, in contrast, will be converted to uracil (and after amplification, thymine). Thus, a differential between the sequencing data for the converted template sequence and the sequencing data for the copy of the template sequence can be used to identify the methylation status of the corresponding cytosine. That is, a substitution (e.g., T in the converted template vs C in the template copy) may be detected between the converted template sequence and the copy of the template sequence, indicating the presence (or absence) of a methylated cytosine at that location in the original template sequence.

Thus, the methods described herein can include generating differential profile data that includes first sequencing data, comprising a nucleic acid sequence corresponding to a template sequence; and second sequencing data, comprising a nucleic acid sequence corresponding to the copy of the template sequence or the copy of the second template sequence, wherein the template sequence and the copy of the template sequence differ by (methylated or unmethylated) cytosine to uracil conversion. The first sequencing data and the second sequencing data may be generated simultaneously (e.g., by including a sequencing primer region 5′ of the template sequence and a copy of the sequencing primer region 5′ of the copy of the template sequence that can simultaneously hybridize to sequencing primers).

The differential profile data may be integrated data that includes the first and second sequencing data (i.e., a single signal at any given flow step that is the sum of the first and second sequencing data).

By way of example, consider a construct with a template sequence TCGTATCTAATGCCATGTA (SEQ ID NO: 5) and an identical copy sequence, except that the nucleic acid construct has been exposed to conditions sufficient to convert unmethylated cytosines in the template sequence to uracils (e.g., the copy sequence is TCGTATCTAACGCCACGTA, SEQ ID NO: 6). As with the example described with respect to Table 2, for each flow step where the template and the copy sequences have the same sequence (i.e., each homopolymer), the expected signal would be twice as intense as compared with the expected signal if only a single sequence (e.g., either the template or the copy) were sequenced. However, at flow steps where the template and the copy sequences differ (e.g., a C to T conversion between the copy and the template sequences), the expected signal would be half as intense as the signal expected if the two sequences were identical. An exemplary non-binary flowgram indicating signal intensity at each flow step (assuming a repeated T-C-G-A flow cycle) is provided in Table 3.

As shown in Table 3, differences between the converted template sequence and the copy of the template sequence can be detected by variations in the observed cumulative signal intensity. Since both the template and the copy sequences are sequenced simultaneously only the cumulative signal will be observable. In this example, for the majority of flow steps, the observed signal will be an even integer (e.g., 0, 2, 4, 6 . . . ). For flow steps where the template and copy sequences differ, the observed signal will be lower (e.g., decreased compared to flow steps where the template and copy sequences are identical). For instance, in flow cycle 5, flow step 1 in Table 3, the observed signal is 1, indicating that primer extension occurred on only one of the template or the copy of the template. Beneficially, sequencing with a repeated T-C-G-A flow cycle can provide information on the sequence of the template and also methylation status of template.

TABLE 3
Example of Expected Signals Detected from Simultaneous Sequencing of Template and Copy Sequences,
Detection of Methylation Sites

| Flow Cycle | 1 | | | | 2 | | | | 3 | | | | 4 | | | | 5 | | | | 6 | | | | 7 | | | | 8 | | | |
|---|---|---|---|---|---|---|---|---|---|---|---|---|---|---|---|---|---|---|---|---|---|---|---|---|---|---|---|---|---|---|---|---|
| Flow Step | 1 | 2 | 3 | 4 | 1 | 2 | 3 | 4 | 1 | 2 | 3 | 4 | 1 | 2 | 3 | 4 | 1 | 2 | 3 | 4 | 1 | 2 | 3 | 4 | 1 | 2 | 3 | 4 | 1 | 2 | 3 | 4 |
| Flow Base | T | C | G | A | T | C | G | A | T | C | G | A | T | C | G | A | T | C | G | A | T | C | G | A | T | C | G | A | T | C | G | A |
| Converted template | 1 | 1 | 1 | 0 | 1 | 0 | 0 | 1 | 1 | 1 | 0 | 0 | 1 | 0 | 0 | 2 | 1 | 0 | 1 | 0 | 0 | 2 | 0 | 1 | 1 | 0 | 1 | 0 | 1 | 0 | 0 | 1 |
| TCGTATCTAATGCCATGT | | | | | | | | | | | | | | | | | | | | | | | | | | | | | | | | |
| A (SEQ ID NO: 5) | | | | | | | | | | | | | | | | | | | | | | | | | | | | | | | | |
| Copy of template | 1 | 1 | 1 | 0 | 1 | 0 | 0 | 1 | 1 | 1 | 0 | 0 | 1 | 0 | 0 | 2 | 0 | 1 | 1 | 0 | 0 | 2 | 0 | 1 | 0 | 1 | 1 | 0 | 1 | 0 | 0 | 1 |
| TCGTATCTAACGCCACGT | | | | | | | | | | | | | | | | | | | | | | | | | | | | | | | | |
| A (SEQ ID NO: 6) | | | | | | | | | | | | | | | | | | | | | | | | | | | | | | | | |
| Observed Signal | 2 | 2 | 2 | 0 | 2 | 0 | 0 | 2 | 2 | 2 | 0 | 0 | 2 | 0 | 0 | 4 | 1 | 1 | 2 | 0 | 0 | 4 | 0 | 2 | 1 | 1 | 2 | 0 | 2 | 0 | 0 | 1 |

The methylation profiling data of a template sequence may include the location of methylated cytosine or non-methylated cytosine in the template sequence. That is, the sequence of the first or second copy of the template sequence can be taken as the ground truth for the sequence of the respective sequence. A thymine base in the sequence of the first (or second) converted template sequence that corresponds to a cytosine base in the first (or second) copy of the template sequence indicates a conversion of a non-methylated cytosine originally found in the first (or second) template sequence if non-methylated cytosine bases were converted to uracil in the conversion reaction. Alternatively, a thymine base in the in the sequence of the first (or second) converted template sequence that corresponds to a cytosine base in the first (or second) copy of the template sequence indicates a conversion of a methylated cytosine originally found in the first (or second) template sequence if methylated cytosine bases were converted to uracil in the conversion reaction. Thus, the methylation profiling data can include a location of methylated cytosine or non-methylated cytosine in the first template sequence or the second template sequence.

In some implementations, the methylation profiling data of a template sequence may include a density or signal intensity of methylated cytosine (or non-methylated cytosine) in the first or second template sequence. That is, it may not be necessary to know the precise locations of the methylated or non-methylated cytosine within the template sequence, but it is sufficient to know what proportion of cytosine bases in the template sequence are methylated. Thus, the first portion or the second portion may be assayed (e.g., by a sequencing process) after conversion to detect signals indicating a conversion of a methylated cytosine to a thymine (or non-methylated cytosine to a thymine).

As discussed above, the sequencing data for determining a nucleic acid sequence (e.g., of a first copy portion or a second copy portion) can include, for each of a plurality of sequencing flow steps, (i) extending the sequencing primer by providing, to the hybridized template, labeled nucleotides of a single base type, and (ii) detecting a signal indicating incorporation of a labeled nucleotide into the extending sequencing primer. While providing nucleotides of a single base type in any given flow step provides accurate sequencing information, the process is relatively slow. Since the precise nucleic acid sequence of the first portion or second portion is not always necessary, described herein is a process for quickly generating methylation status data.

Methylated cytosine bases most frequently occur within CpG sites. Thus, a single cytosine (i.e., not flanked by a cytosine) in the template is considered unlikely to be methylated in the original template sequence, although may be residual from incomplete conversion (e.g., the non-methylated cytosine was not converted to uracil because the reaction did not go to completion). By labeling the cytosine bases (rather than guanine bases), no detectable signal is produced due to an isolated cytosine. But including a mixture of cytosine and guanine bases, wherein at least a portion of the cytosine bases are labeled, CpG sites (where the cytosine base remains unconverted) will provide a detectable signal due to incorporation of the labeled cytosine nucleotide resulting from the presence of a G in the template strand.

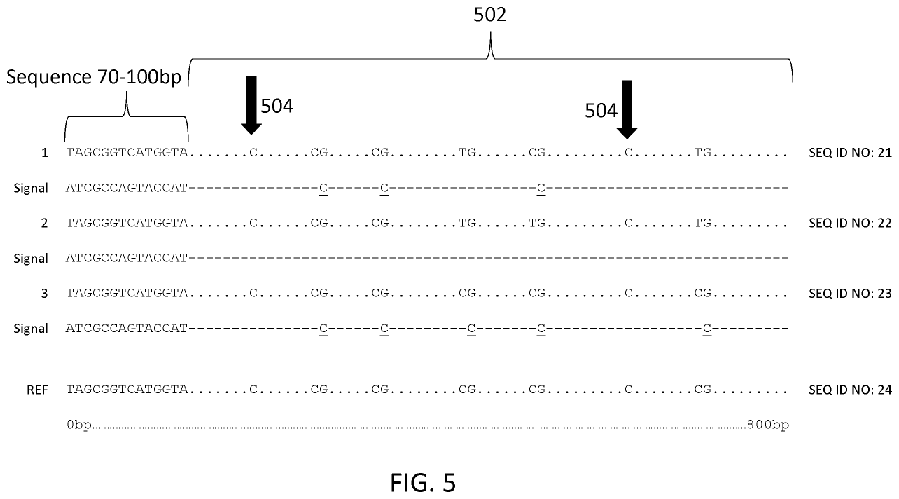

FIG. 5 illustrates exemplary methylation status data that may be obtained using the methods described herein. The illustrated examples show three identical nucleic acid sequences (aligned with a reference sequence), except the methylation profile differs between the sequences within the 502 regions. Below each sequence is the respective signal that may be detected by flowing a complementary labeled nucleotide in a flow sequencing process. The first 70-100 bases of the nucleic acid molecule are sequenced using a standard flow sequencing cycles, wherein a single base type is discretely provided in each sequencing flow step (e.g., a flow cycle of T-G-C-A). It will be appreciated that any other number of bases may be sequenced using the standard flow cycles (e.g., about 0, 10, 20, 30, 40, 50, 60, 70, 80, 90, 100, 120, 150, 200 etc. bases). In some cases, this initial portion of the sequence read can be used to map the sequence read to a portion of a reference genome. For the remainder of the sequencing flow cycles (or for a predetermined number of flow cycles), a first flow step provides unlabeled T, C, and A nucleotides, and a second flow step provides unlabeled G and labeled C nucleotides. Thus, the first flow step will enable extension of the sequencing primer molecule until a cytosine residue is present in the template sequence (and/or the copy of the template sequence). The second flow step will enable extension of the sequencing primer molecule until a thymine or adenine is present in the template sequence (and/or the copy of the template sequence). No signals will be detected in any of the first flow steps (e.g., due to the lack of any labeled nucleotides). Indeed, no detection step need be performed following a first flow step (thus increasing efficiency of sequencing). For a second flow step, any CpG sites present in the template or copy of the template sequence will result in detectable signal (e.g., due to incorporation of a labeled C), and primer extension through unmethylated CpG 504 sites will not result in detectable signal (e.g., due to lack of incorporation of a labeled C).

It will be appreciated that different combinations of labeled and unlabeled nucleotides may be used in the first and second flow steps described above. For example, one or more of the nucleotide bases in the first flow step may be labeled, and incorporation of such nucleotides may be detected.

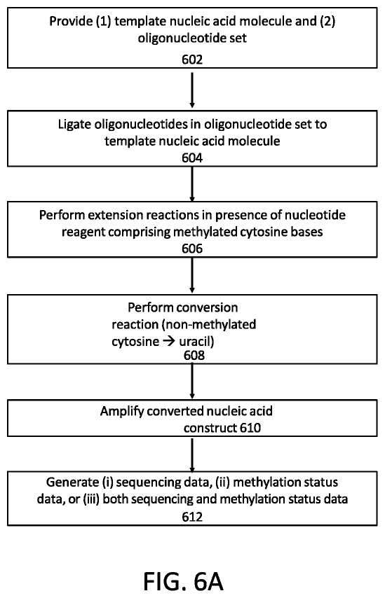

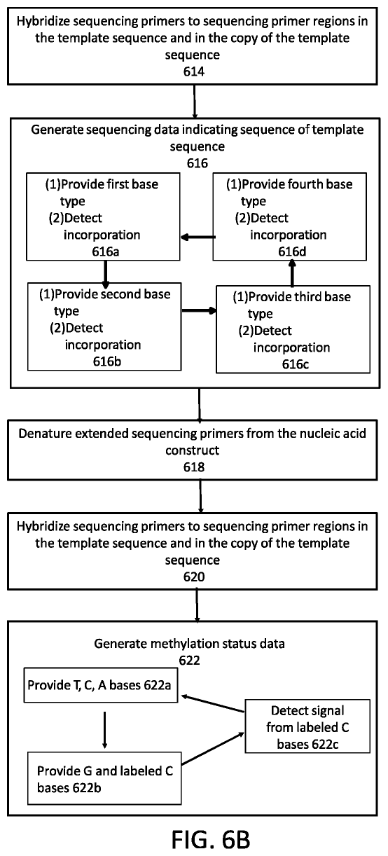

FIG. 6A illustrates an exemplary method for obtaining sequencing data and/or methylation status data for a nucleic acid molecule. At 602, a template nucleic acid molecule and an oligonucleotide set are provided. The template nucleic acid molecule is a duplex molecule with a “top” strand and a “bottom” strand. The oligonucleotide set includes four oligonucleotides, portions of which hybridize (e.g., through reverse complementarity) to form a complex comprising the four-oligonucleotides. The oligonucleotide set, as described elsewhere herein, can assemble such that a 3′ portion of the first oligonucleotide hybridizes to a 5′ portion of the second oligonucleotide, a 3′ portion of the second oligonucleotide hybridizes to a 3′ portion of the third oligonucleotide, and a 5′ portion of the third oligonucleotide hybridizes to a 3′ portion of the fourth oligonucleotide. The first oligonucleotide may further include a 5′ portion that includes a sequencing primer region (e.g., includes a hybridization site for a sequencing primer).

The oligonucleotide set is then ligated to the template nucleic acid at 604. For example, a 3′ terminus of the first oligonucleotide can be ligated to a 5′ terminus of the first strand of the template nucleic acid, a 5′ terminus of the second oligonucleotide can be ligated to a 3′ terminus of the second strand, a 5′ terminus of the third oligonucleotide can be ligated to a 3′ terminus of the first strand, and a 3′ terminus of the fourth oligonucleotide can be ligated to a 5′ terminus of the second strand.

At 606, extension reactions are performed in the presence of a nucleotide reagent comprising methylated cytosine bases. Substantially all cytosine bases in the nucleotide reagent may be methylated cytosine bases. The nucleotide regent also includes other nucleotides necessary for the extension reaction (e.g., A, T, and G bases). The resulting construct includes a first strand comprising the first template sequence portion (“original top”) and a first copy portion (“copied top”), and a second strand comprising the second template sequence portion (“original bottom”) and a second copy portion (“copied bottom”).

At 608 the construct subjected to a conversion reaction, which converts non-methylated cytosine to uracil, thereby forming a converted nucleic acid construct (e.g., where the template sequence has been converted). The converted nucleic acid construct is amplified at 510, which replaces uracil bases with thymine bases in the amplified product (e.g., in the copy of the template sequence).

At 612, sequencing data, methylation status data, or both may be obtained from the converted nucleic acid construct. FIG. 6B provides further detail for obtaining methylation profiling data in accordance with some embodiments. At 614, sequencing primers are hybridized to sequencing primer regions of the converted nucleic acid molecule. At 616, sequencing data is generated, concurrently from. The sequencing data is generated using a plurality of sequencing flow steps in a flow cycle order. The primers are extended as the sequencing data is generated. In each flow step, labeled nucleotides of a single base type are provide to the hybridized template, followed by detecting a signal indicating incorporation of a labeled nucleotide into the extending sequencing primers. Differential sequencing signals may be detected as described herein. At 618, the extended sequencing primers are removed from the converted nucleic acid molecule (e.g., chemical, thermal, enzymatic degradation). At 620, sequencing primers are hybridized to sequencing primer regions of the converted nucleic acid molecule. At 622 methylation status data is generated for the converted template portion. The sequencing primers are further extended as the methylation status data is generated. In a first flow step, a mixture of thymine, cytosine and adenine bases are provided to the hybridized template at 622a; primer extension stalls when a cytosine base is present in the template sequence and/or in the copy of the template sequence. In a second flow step, guanine and cytosine bases, wherein at least a portion of the cytosine bases are labeled, are then provided at 622b. At 622c, incorporation of labeled C bases is detected in the template sequence and/or the copy of the template sequence; the differential signal detected at a locus where the template sequence comprises a T and the copy of the template sequence comprises a C (or the reverse) indicates the methylation status of the original template molecule.

By way of example, consider again a construct with a template sequence TCGTATCTAATGCCATGTA (SEQ ID NO: 5) and an identical copy sequence, except that the nucleic acid construct has been exposed to conditions sufficient to convert unmethylated cytosines in the template sequence to uracils (e.g., the copy sequence is TCGTATCTAACGCCACGTA, SEQ ID NO: 6). An exemplary non-binary flowgram indicating signal intensity at each flow step (assuming a repeated TGA-CG flow cycle, where C is labeled in each flow step 2) is provided in Table 4.

TABLE 4
Example of Expected Signals Detected from Simultaneous Sequencing of Template and Copy
Sequences, Detection of Methylation Status

| Flow cycle | 1 | | 2 | | 3 | | 4 | | 5 | |
|---|---|---|---|---|---|---|---|---|---|---|
| Flow step | 1 | 2 | 1 | 2 | 1 | 2 | 1 | 2 | 1 | 2 |
| Flow bases | TCA | CG | TCA | CG | TCA | CG | TCA | CG | TCA | CG |
| Converted Template\| | — | 1 | — | 0 | — | 0 | — | 0 | — | 0 |
| TCGTATCTAATGCCATGTA (SEQ ID NO: 5) | | | | | | | | | | |
| Copy of Template\| | — | 1 | — | 0 | — | 1 | — | 1 | — | 0 |
| TCGTATCTAACGCCACGTA (SEQ ID NO: 6) | | | | | | | | | | |
| Total Fluorescence Observed | — | 2 | — | 0 | — | 1 | — | 1 | — | 0 |
| Bases sequenced per flow step | | | | | | | | | | |
| (e.g., where does primer extension | | | | | | | | | | |
| stall in each sequence in the flow | | | | | | | | | | |
| step) | | | | | | | | | | |
| Converted Template\| | T | CG | TAT | C | TAATG | CC | ATGTA | — | — | — |
| TCGTATCTAATGCCATGTA (SEQ ID NO: 5) | | | | | | | | | | |
| Copy of Template\| | T | CG | TAT | C | TAA | CGCC | A | CG | TA | — |
| TCGTATCTAACGCCACGTA (SEQ ID NO: 6) | | | | | | | | | | |

As shown in Table 4, differences between the converted template sequence and the copy of the template sequence can be detected by variations in the observed cumulative signal intensity. Since both the template and the copy sequences are sequenced simultaneously only the cumulative signal will be observable. Primer extension in each sequences will stall during TCA flow steps wherever a cytosine base is present (e.g., due to the lack of guanines); and congruently, primer extension in each sequence will proceed until a cytosine base (e.g., any number of non-guanine bases may be incorporated prior to primer extension stalling). The next flow step of G and labeled C nucleotides interrogates whether a CpG site is present or not (e.g., whether a guanine follows the cytosine that prompted the stalling of primer extension). In this example, for GC flow steps where the template sequence and the copy of the template include the same number of guanine residues, the observed signal will be an even integer (e.g., 0, 2, 4, 6 . . . ). See for example, flow cycle 1, flow step 2 in Table 4, where the observed signal is 2. For GC flow steps where the template and copy sequences differ (e.g., indicating a methylation site in the unconverted template sequence), the observed signal will be lower (e.g., decreased compared to flow steps where the template and copy sequences are identical). For instance, in flow cycle 3, flow step 2 in Table 4, the observed signal is 1, indicating that primer extension occurred on only one of the template or the copy of the template. Beneficially, sequencing with a repeating TGA-GC flow cycle will more efficiently provide methylation density information for the template sequence (e.g., will require fewer flow steps than if using a repeating T-G-C-A or other four nucleotide flow cycle). In addition, as with other sequencing methods described herein, the concurrent sequencing of the converted template and the copy of the template can provide higher confidence in the determined sequence (and/or methylation status data).

It will be appreciated that the steps in FIG. 6B may be performed in a different order (e.g., methylation status data generated prior to sequencing data), or some steps in FIG. 6B may be omitted altogether (e.g., only methylation status data or sequencing data may be generated), depending upon the desired data to be obtained from sequencing the nucleic acid construct.

Repeat Sequencing for Variant Detection and/or Methylation Status and Sequencing Data

In some cases, both the full sequence of a template and the methylation status of the template may be desired. In such cases, the methods described herein may be applied sequentially (e.g., for at least two flow orders, at least 3 flow orders, at least four flow orders, etc.). For example, a first flow order comprising A, C, G, and T nucleotides (e.g., as described with respect to Table 2) may be used to determine the nucleotide sequence of the template sequence. That is, primer extension through the template sequence and through the copy of the template sequence may be performed concurrently using the first flow order. After this primer extension (e.g., through substantially the entire length of the template sequence (and the copy of the template sequence)), the resulting double stranded molecule comprising the nucleic acid construct may be denatured (e.g., via chemical means, enzymatic degradation, exposure to heat) and subsequently reannealed to primer molecules (e.g., using sequencing primers that anneal to the same sequencing primer regions). Then a second flow order (e.g., as described with respect to Table 3) may be used to determine methylation status of the template sequence (e.g., by concurrent primer extension through the template sequence and the copy of the template sequence). It will be appreciated that methylation sequencing may be performed prior to nucleotide base sequencing (e.g., the second flow order may be used before the first flow order). Additionally, it will be understood that additional flow orders to those described herein may be used.

As an example, sequencing primers can be hybridized to the sequencing primer region 5′ of the template sequence and to the sequencing primer region 5′ of the copy of the template sequence (e.g., to both sequencing primer regions of the nucleic acid construct) to form a hybridized template. First sequencing data can be generated by, for each of a plurality of sequencing flow steps according to a first flow order, (i) extending the sequencing primers in the template and the copy of the template respectively by providing, to the hybridized template, labeled nucleotides of a single base type, and (ii) detecting a signal indicating incorporation of a labeled nucleotide into the extending sequencing primers. Second sequencing data can also be generated by, for each of a plurality of sequencing flow steps according to a second flow order, (i) extending the sequencing primer by providing, to the hybridized template, labeled nucleotides of a single base type, and (ii) detecting a signal indicating incorporation of a labeled nucleotide into the extending sequencing primer. The first flow order and the second flow order are different so that the resulting sequencing data is different. Different flow orders can result in different sensitivities for different contextual variants. A variant missed using the first flow order may be detected using the second flow order.

### Exemplary Embodiments

The following embodiments are exemplary and are not intended to limit the scope of the invention described herein.

Embodiment 1. A composition, comprising:
an oligonucleotide set, comprising a first oligonucleotide, a second oligonucleotide, a third oligonucleotide, and a fourth oligonucleotide, wherein:
a 3′ portion of the first oligonucleotide is hybridized to a 5′ portion of the second oligonucleotide, wherein 3′ portion of the first oligonucleotide comprises a sequencing primer region,
a 3′ portion of the second oligonucleotide is hybridized to a 3′ portion of the third oligonucleotide, and
a 5′ portion of the third oligonucleotide is hybridized to a 3′ portion of the fourth oligonucleotide.

Embodiment 2. The composition of embodiment 1, wherein:
a 3′ terminus of the first oligonucleotide is coupled to a 5′ terminus of a first strand of a template nucleic acid molecule,
a 5′ terminus of the second oligonucleotide is coupled to a 3′ terminus of a second strand of the template nucleic acid molecule, wherein the second strand is hybridized to the first strand,
a 5′ terminus of the third oligonucleotide is coupled to a 3′ terminus of the first strand, and
a 3′ terminus of the fourth oligonucleotide is coupled to a 5′ terminus of the second strand.

Embodiment 3. The composition of embodiment 1, wherein:
the first strand of the template nucleic acid molecule comprises a first template sequence; and
the second strand of the template nucleic acid molecule comprises a second template sequence, wherein the second template sequence is a reverse complement of the first template sequence.

Embodiment 4. The composition of any one of embodiments 1-3, wherein 3′ portion of the first oligonucleotide comprises a barcode sequence positioned between the sequencing primer region and the 3′ terminus of the first oligonucleotide.

Embodiment 5. The composition of any one of embodiments 1-4, wherein a 5′ portion of the first oligonucleotide comprises a forward amplification primer region, and a 5′ portion of the fourth oligonucleotide comprises a reverse amplification primer region.

Embodiment 6. A composition, comprising:
a nucleic acid molecule comprising a first construct strand and a second construct strand, wherein:
the first construct strand comprises, from 5′ to 3′, a first sequencing primer region, a first template sequence, a first nucleic acid linker, a copy of the first sequencing primer region, and a single copy of the first template sequence; and
the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a single copy of the second template sequence, and a copy of the second sequencing primer region.

Embodiment 7. A composition, comprising:
a nucleic acid molecule comprising a first construct strand and a second construct strand, wherein:
the first construct strand comprises, from 5′ to 3′, a first sequencing primer region, a first template sequence, a first nucleic acid linker, a copy of the first sequencing primer region, wherein substantially all cytosine bases in the copy of the first sequencing primer region are methylated, and a copy of the first template sequence, wherein substantially all cytosine bases in the copy of the first template sequence are methylated; and the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, wherein substantially all cytosine bases in the copy of the second template sequence are methylated, and a copy of the second sequencing primer region, wherein substantially all cytosine bases in the copy of the second sequencing primer region are methylated.

Embodiment 8. A method, comprising:
(a) providing:
a template nucleic acid molecule comprising a first strand comprising a first template sequence and a second strand comprising a second template sequence, wherein the first template sequence is a reverse complement of the second template sequence and wherein the first strand is hybridized to the second strand; and an oligonucleotide set, comprising a first oligonucleotide, a second oligonucleotide, a third oligonucleotide, and a fourth oligonucleotide, wherein:
a 3′ portion of the first oligonucleotide hybridizes to a 5′ portion of the second oligonucleotide, wherein 3′ portion of the first oligonucleotide comprises a sequencing primer region,
a 3′ portion of the second oligonucleotide hybridizes to a 3′ portion of the third oligonucleotide, and
a 5′ portion of the third oligonucleotide hybridizes to a 3′ portion of the fourth oligonucleotide; and
(b) ligating:
a 3′ terminus of the first oligonucleotide to a 5′ terminus of the first strand,
a 5′ terminus of the second oligonucleotide to a 3′ terminus of the second strand,
a 5′ terminus of the third oligonucleotide to a 3′ terminus of the first strand, and
a 3′ terminus of the fourth oligonucleotide to a 5′ terminus of the second strand.

Embodiment 9. The method of embodiment 8, further comprising crosslinking the second oligonucleotide to the third oligonucleotide.

Embodiment 10. The method of embodiment 9, wherein the crosslinking is a reversible crosslinking.

Embodiment 11. The method of embodiment 9 or 10, wherein the second oligonucleotide is crosslinked to the third oligonucleotide before the ligating.

Embodiment 12. The method of embodiment 9 or 10, wherein the second oligonucleotide is crosslinked to the third oligonucleotide after the ligating.

Embodiment 13. The method of any one of embodiments 8-12, wherein the method further comprises:
(c) performing extension reactions, comprising:
extending the 3′ terminus of the second oligonucleotide using, in order, a portion of the third oligonucleotide, the first strand, and the first oligonucleotide as a template,
extending the 3′ terminus of the third oligonucleotide using, in order, a portion of the second oligonucleotide, the second strand, and the fourth oligonucleotide as a template.

Embodiment 14. The method of embodiment 13, wherein the extension reactions generate a nucleic acid molecule comprising a first construct strand and a second construct strand, wherein:
the first construct strand comprises, from 5′ to 3′, a first sequencing primer region, a first template sequence, a first nucleic acid linker, a copy of the first sequencing primer region, and a copy of the first template sequence; and
the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, and a copy of the second sequencing primer region.

Embodiment 15. The method of embodiment 14, wherein the extension reactions are performed in the presence of a nucleotide reagent comprising methylated cytosine bases and substantially no unmethylated cytosine bases, and wherein substantially all cytosine bases in the copy of the first sequencing primer region, the copy of the first template sequence, the copy of the sequence sequencing primer region, and the copy of the second template sequence are methylated.

Embodiment 16. The method of embodiment 15, wherein the sequencing primer region is free of cytosine bases.

Embodiment 17. The method of any one of embodiments 14-16, wherein the first nucleic acid linker or the second nucleic acid linker is about 30 bases in length to about the length of the first template sequence or the second template sequence.

Embodiment 18. The method of any one of embodiments 14-17, wherein the first nucleic acid linker or the second nucleic acid linker is between about 20% and about 100% of a length of the first template sequence or the second template sequence.

Embodiment 19. The method of any one of embodiments 14-18, wherein the first nucleic acid linker and the second nucleic acid linker each have a known sequence.

Embodiment 20. The method of any one of embodiments 8-19, wherein 3′ portion of the first oligonucleotide further comprises a barcode sequence.

Embodiment 21. The method of embodiment 20, wherein the barcode sequence is positioned between the sequencing primer region and the 3′ terminus of the first strand.

Embodiment 22. The method of embodiment 21, wherein the barcode sequence comprises a unique molecular identifier.

Embodiment 23. The method of embodiment 21 or 22, wherein the barcode sequence comprises a sample barcode.

Embodiment 24. The method of any one of embodiments 0, wherein 3′ portion of the first oligonucleotide further comprises a preamble sequence.

Embodiment 25. The method of embodiment 24, wherein the preamble sequence is positioned between the sequencing primer region and the 3′ terminus of the first strand.

Embodiment 26. A nucleic acid molecule construct made according to the method of any one of embodiments 8-25.

Embodiment 27. The method of any one of embodiments 8-26, further comprising generating differential profile data comprising:
first sequencing data, comprising a nucleic acid sequence corresponding to the first template sequence or the second template sequence; and
second sequencing data, comprising a nucleic acid sequence corresponding to the copy of the first template sequence or the copy of the second template sequence.

Embodiment 28. The method of embodiment 27, wherein the first sequencing data and the second sequencing data of the differential profile data are obtained from a same first strand sequencing read or a same second strand sequencing read.

Embodiment 29. The method of embodiment 27 or 28, further comprising filtering library sequencing data to remove sequencing reads associated with a difference between the first sequencing data and the second sequencing data.

Embodiment 30. The method of embodiment 27 or 28, wherein the differential profile data comprises methylation data, and the method further comprises identifying a location of one or more methylated or unmethylated cytosine residues in the template nucleic acid molecule.

Embodiment 31. The method of any one of embodiments 27-30, further comprising generating sequencing data, comprising:
hybridizing sequencing primers to the first sequencing primer region and to the copy of the first sequencing primer region on the first construct strand to form a hybridized template; and
generating sequencing data from the first template sequence and the first copy portion simultaneously, comprising extending the sequencing primers by, for each of a plurality of sequencing flow steps, (i) providing, to the hybridized template, labeled nucleotides of a single base type, and (ii) detecting a signal indicating incorporation of a labeled nucleotide into the extending sequencing primers.

Embodiment 32. The method of any one of embodiments 27-31, further comprising generating sequencing data, comprising:
hybridizing sequencing primers to the second sequencing primer region and to the copy of the second sequencing primer region on the second construct strand to form a hybridized template; and
generating sequencing data from the second template sequence and the second copy portion simultaneously, comprising extending the sequencing primers by, for each of a plurality of sequencing flow steps, (i) providing, to the hybridized template, labeled nucleotides of a single base type, and (ii) detecting a signal indicating incorporation of a labeled nucleotide into the extending sequencing primers.

Embodiment 33. A method, comprising:
(a) providing:
a template nucleic acid molecule comprising a first strand comprising a first template sequence and a second strand comprising a second template sequence, wherein the first template sequence is a reverse complement of the second template sequence; and
an oligonucleotide set, comprising a first oligonucleotide, a second oligonucleotide, a third oligonucleotide, and a fourth oligonucleotide, wherein:
a 3′ portion of the first oligonucleotide hybridizes to a 5′ portion of the second oligonucleotide, wherein 3′ portion of the first oligonucleotide comprises a first sequencing primer region,
a 3′ portion of the second oligonucleotide hybridizes to a 3′ portion of the third oligonucleotide, and
a 5′ portion of the third oligonucleotide hybridizes to a 3′ portion of the fourth oligonucleotide;
(b) ligating:
a 3′ terminus of the first oligonucleotide to a 5′ terminus of the first strand,
a 5′ terminus of the second oligonucleotide to a 3′ terminus of the second strand,
a 5′ terminus of the third oligonucleotide to a 3′ terminus of the first strand, and
a 3′ terminus of the fourth oligonucleotide to a 5′ terminus of the second strand;
(c) performing extension reactions in the presence of a nucleotide reagent comprising methylated cytosine bases, comprising:
extending the 3′ terminus of the second oligonucleotide using, in order, a portion of the third oligonucleotide, the first strand, and the first oligonucleotide as a template,
extending the 3′ terminus of the third oligonucleotide using, in order, a portion of the second oligonucleotide, the second strand, and the fourth oligonucleotide as a template,
thereby generating a nucleic acid molecule comprising a first construct strand and a second construct strand, wherein:
the first construct strand comprises, from 5′ to 3′, a first sequencing primer region, a first template sequence, a first nucleic acid linker, a copy of the first sequencing primer region, and a copy of the first template sequence; and
the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker, wherein the second nucleic acid linker is a reverse complement of the first nucleic acid linker, a copy of the second template sequence, and a copy of the second sequencing primer region;
(d) sequencing the first strand, comprising:
hybridizing sequencing primers to the first sequencing primer region and to the copy of the first sequencing primer region on the first strand, or to the second sequencing primer region and to the copy of the second sequencing primer region on the second strand, to form a hybridized template;
generating first sequencing data for the copy of the first template sequence or the copy of the second template sequence, comprising extending the sequencing primers by, for each of a plurality of sequencing flow steps according to a first flow order, (i) providing, to the hybridized template, labeled nucleotides of a single base type, and (ii) detecting a signal indicating incorporation of a labeled nucleotide into the extending sequencing primers; and
generating second sequencing data for the first template sequence or the second template sequence, comprising extending the sequencing primers by, for each of a plurality of sequencing flow steps according to a second flow order, (i) providing, to the hybridized template, labeled nucleotides of a single base type, and (ii) detecting a signal indicating incorporation of a labeled nucleotide into the extending sequencing primers,
wherein the first sequencing data and the second sequencing data are generated simultaneously.

### Examples

### Example 1: Sequences for the Oligonucleotide Set

As described elsewhere herein, there may be advantages to selecting sequences for the oligonucleotides in the oligonucleotide set for generating a nucleic acid construct. Some sequences may be advantageous for a specific type of sequencing (e.g., methylation vs non-methylation sequencing). Some sequences may be advantageous for specific types of flow orders (e.g., for flow based sequencing). Some sequences may provide advantageous structural features (e.g., inducing curvature, reducing unwanted secondary structures, etc.).

In Tables 5, 6, and 7 below, reference is made to the different portions of Oligonucleotide set 202 as illustrated in FIG. 2. * indicates a phosphorylated nucleotide base. NNN indicates any nucleotide base type or types and may serve as a UMI. It will be appreciated that the NNN regions may be a different number of nucleotide bases (e.g., 2, 4, 5, 6, 7, 8, 9, 10 bases) in length. For example, in some instances, a UMI may consist of NN or NNNN. In some cases, there may not be a need for a UMI, and therefore the NNN regions may be removed entirely (see e.g., Tables 6 and 7 where SEQ ID NOS. 12, 13, 16, and 17 lack NNN regions). In some instances, a barcode sequence or other type of sequence (e.g., an adapter, a primer hybridization region, a preamble sequence, etc.) may be additionally included in the sequences in Tables 5, 6, and 7 (e.g., appended to or integrated within).

The oligo sequences in Table 5 are not optimized to any particular flow order and serve as reference sequences with which to compare the sequences in Tables 6 and 7. Flow order optimization may not be required for some types of sequencing or in cases where the overall length of the nucleic acid construct molecule is not of concern. A nucleic acid construct molecule resulting from the sequences listed in Table 5 will be:

(SEQ ID NO: 7)
ACCATCTCATCCCTGCGTGTCTCCGACTGCACACATCCTGCATGTGAT
-Template sequence-
(SEQ ID NO: 19)
GTAGTCTAACGCTCGGTGNNNCAGATGTACGACAATGATCACTTAGTCA
CTTATTGGGTCACGGTGTGGCTTCGAGGATCAACACGTCAGAGTCTAGC
GCCAATCCGTTCTGAGCTCTACGACCGACAGTGACGGTGGACTATNNNT
GCACACATCCTGCATGTGA
-Copy of Template sequence-
(SEQ ID NO: 20)
GTAGTCTAACGCTCGGTGATCACCGACTGCCCATAGAGAGCTGAG

In some cases, e.g., for flow-based sequencing, it may be desirable to optimize the non-template-based portions of a nucleic acid construct molecule for a particular flow order. Beneficially, this optimization may reduce the number of flow cycles required to extend a sequencing primer through these regions of the nucleic acid construct (e.g., the non-template regions). This will increase the efficiency of sequencing. It will be appreciated that different sequences will be optimized for different flow orders (e.g., a T-G-C-A flow order vs a T-C-A-G flow order). In Tables 6 and 7 below, the oligonucleotides are optimized such that at least a single base is incorporated into an extending primer per flow step. The sequences in Table 7 may advantageously exhibit reduced secondary structure formation during assembly of a nucleic acid construct in accordance with methods described herein.

TABLE 5
Example Oligonucleotide Set Sequences.

| Oligonucleotide | | |
|---|---|---|
| (as indicated | | |
| in FIG. 2) | Sequence | SEQ ID NO. |
| 204 | ACCATCTCATCCCTGCGTGTCTCCGACTGCACACATCCTGCATGTGAT | SEQ ID NO: 8 |
| 206 | TCACATGCAGGATGTGTGCANNNATAGTCCACCGTCACTGTCGGTCGTAGAGCTC | SEQ ID NO: 9 |
| | AGAACGGATTGGCGCTAGACTCTGACGTGTTGATCCTCGAAGC | |
| 208 | GTAGTCTAACGCTCGGTGNNNCAGATGTACGACAATGATCACTTAGTCACTTATT | SEQ ID NO: 10 |
| | GGGTCACGGTGTGGCTTCGAGGATCAACACGTCAGAGTCTAGC | |
| 210 | CTCAGCTCTCTATGGGCAGTCGGTGATCACCGAGCGTTAGACTAC*T | SEQ ID NO: 11 |

TABLE 6
Oligonucleotide Set Sequences, optimized for a T-G-C-A flow order.

| Oligonucleotide | | |
|---|---|---|
| (as indicated | | |
| in FIG. 2) | Sequence | SEQ ID NO. |
| 204 | ACCATCTCATCCCTGCGTGTCTCCGACTGCACACATCCTGCATGTGAT | SEQ ID NO: 8 |
| 206 | TCACATGCAGGATGTGTGCAGGAAAATCCCCCCCATGGCAGTGCAAACATGCAAA | SEQ ID NO: 12 |
| | AGGCATTGGCGCTAGACTTTGGGCTTTTGACCTTGCAAGC | |
| 208 | GATGGGAATTGGCCCTGGCAATGTGCAAGCTTTCCTTTTTTGTTCATTTTTCCCCAT | SEQ ID NO: 13 |
| | TCGGGTGGCTTGCAAGGTCAAAAGCCCAAAGTCTAGC | |
| 210 | CTCAGCTCTCTATGGGCAGTCGGTGATCAGGGCCAATTCCCATCA*T | SEQ ID NO: 14 |

TABLE: 7
Example Oligonucleotide Set Sequences, optimized for a T-G-C-A flow order and decreased
secondary structure.

| Oligonucleotide | | |
|---|---|---|
| (as indicated | | |
| in FIG. 2) | Sequence | SEQ ID NO. |
| 204 | TCCATCTCATCCCTGCGTGTCTCCGACTGCACACATCCTGCATGTGAT | SEQ ID NO: 15 |
| 206 | TCACATGCAGGATGTGTGCATCCAAATCCCCCCCATGGCAGTGCAAAGATGCAAA | SEQ ID NO: 16 |
| | AGGCATTGGCGCTGCTAGACTTTGGGATTTTGACCTTGAAAGC | |
| 208 | TGATGGGAATTGGCCCTGGCAATGTGGAAGCTTTCCTTTTTTGTTCATTTTTCCCCA | SEQ ID NO: 17 |
| | TTCGGGTGGCTTTCAAGGTCAAAATCCCAAAGTCTAG*C | |
| 210 | CTCAGCTCTCTATGGGCAGTCGGTGATCAGGGCCAATTCCCATCA*T | SEQ ID NO: 18 |

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1.-33. (canceled)

34. A composition, comprising:
a nucleic acid molecule comprising a first construct strand and a second construct strand,
wherein:
the first construct strand comprises, from 5′ to 3′, a first sequencing primer region, a first template sequence, a first nucleic acid linker, a copy of the first sequencing primer region, and a single copy of the first template sequence; and
the second construct strand comprises, from 5′ to 3′, a second template sequence, wherein the second template sequence is a reverse complement of the first template sequence, a second sequencing primer region,
wherein the second sequencing primer region is a reverse complement of the first sequencing primer region, a second nucleic acid linker, and
wherein the second nucleic acid linker is a reverse complement of the first nucleic acid linker, a single copy of the second template sequence, and a copy of the second sequencing primer region.

35. The composition of claim 34, wherein the first sequencing primer region or the copy of the first sequencing primer region is free of cytosine bases.

36. The composition of claim 34, wherein the single copy of the first template sequence is a reverse complement of the second template sequence.

37. The composition of claim 34, wherein the first nucleic acid linker or the second nucleic acid linker is about 30 bases in length to about (1) a length of the first template sequence or (2) a length of the second template sequence.

38. The composition of claim 34, wherein the first nucleic acid linker or the second nucleic acid linker is from about 20% to about 100% of (1) a length of the first template sequence or (2) a length of the second template sequence.

39. The composition of claim 34, wherein the first nucleic acid linker and the second nucleic acid linker each comprises a known sequence.

40. The composition of claim 34, wherein the first construct strand comprises a first barcode sequence associated with the first template sequence and a copy of the first barcode sequence associated with the copy of the first template sequence, and wherein the second construct strand comprises a second barcode sequence associated with the second template sequence and a copy of the second barcode sequence associated with the copy of the second template sequence.

41. The composition of claim 40, wherein the first barcode sequence is positioned between the first sequencing primer region and the first template sequence, and wherein the second barcode sequence is positioned between the second sequencing primer region and the second template sequence.

42. The composition of claim 40, wherein the first barcode sequence or the second barcode sequence comprises a unique molecular identifier.

43. The composition of claim 34, wherein the first construct strand further comprises a preamble sequence.

44. The composition of claim 43, wherein the preamble sequence is positioned between the first sequencing primer region and a 3′ terminus of the first construct strand.

45. A composition, comprising:
a nucleic acid molecule comprising a first construct strand and a second construct strand, wherein:
the first construct strand comprises, from 5′ to 3′, a first sequencing primer region, a first template sequence, a first nucleic acid linker, a copy of the first sequencing primer region,
wherein substantially all cytosine bases in the copy of the first sequencing primer region are methylated, and a copy of the first template sequence,
wherein substantially all cytosine bases in the copy of the first template sequence are methylated; and
the second construct strand comprises, from 5′ to 3′, a second template sequence, wherein the second template sequence is a reverse complement of the first template sequence, a second sequencing primer region, wherein the second sequencing primer region is a reverse complement of the first sequencing primer region, a second nucleic acid linker,
wherein the second nucleic acid linker is a reverse complement of the first nucleic acid linker, a single copy of the second template sequence,
wherein substantially all cytosine bases in the copy of the second template sequence are methylated, and a copy of the second sequencing primer region, and
wherein substantially all cytosine bases in the copy of the second sequencing primer region are methylated.

46. The composition of claim 45, wherein the first sequencing primer region or the second sequencing primer region is free of cytosine bases.

47. The composition of claim 45, wherein the first nucleic acid linker or the second nucleic acid linker is about 30 bases in length to about (1) a length of the first template sequence or (2) a length of the second template sequence.

48. The composition of claim 45, wherein the first nucleic acid linker or the second nucleic acid linker is from about 20% to about 100% of (1) a length of the first template sequence or (2) a length of the second template sequence.

49. The composition of claim 45, wherein the first nucleic acid linker and the second nucleic acid linker each comprises a known sequence.

50. The composition of claim 45, wherein the first construct strand comprises a first barcode sequence associated with the first template sequence and a copy of the first barcode sequence associated with the copy of the first template sequence, and wherein the second construct strand comprises a second barcode sequence associated with the second template sequence and a copy of the second barcode sequence associated with the copy of the second template sequence.

51. The composition of claim 50, wherein the first barcode sequence is positioned between the first sequencing primer region and the first template sequence, and wherein the second barcode sequence is positioned between the second sequencing primer region and the second template sequence.

52. The composition of claim 45, wherein the first construct strand further comprises a preamble sequence.

53. The composition of claim 52, wherein the preamble sequence is positioned between the first sequencing primer region and a 3′ terminus of the first construct strand.
